SEQUENCE LISTING (26 sequences)

SEQ ID NO: 1 - DNA, 29 bp, Artificial
  ATHK1-specific primer 1
aggaaggtgttcgataaaatgactgaatg

SEQ ID NO: 2 - DNA, 28 bp, Artificial
  ATHK1-specific primer 2
cacatccagtatcatcaacctcaaacca

SEQ ID NO: 3 - DNA, 29 bp, Artificial
  T-DNA border primer 1
cattttataataacgctgcggacatctac

SEQ ID NO: 4 - DNA, 29 bp, Artificial
  T-DNA border primer 2
tttctccatattgaccatcatactcattg

SEQ ID NO: 5 - DNA, 36 bp, Artificial
  6 kb ATHK1 fragment, primer 1
ccgctcgagctctccattggccattttaccttctac

SEQ ID NO: 6 - DNA, 41 bp, Artificial
  6 kb ATHK1 fragment, primer 2
ataagaatgcggccgcttacccccaaaaacctcatcgtcaa

SEQ ID NO: 7 - DNA, 36 bp, Artificial
  8 kb ATHK1 fragment, primer 1
ccgctcgagtgttaaatcgcagtctatacagtcatc

SEQ ID NO: 8 - DNA, 42 bp, Artificial
  8 kb ATHK1 fragment, primer 2
ataagaatgcggccgcgggcttaaaaattgttccagagttcg

SEQ ID NO: 9 - DNA, 29 bp, Artificial
  ATHK1 forward primer for QRT-PCR
ctagtttacggaattcctcgccttatgac

SEQ ID NO: 10 - DNA, 29 bp, Artificial
  ATHK1 reverse primer for QRT-PCR
ctctgtgccaatctctgctctccttatcg

SEQ ID NO: 11 - DNA, 21 bp, Artificial
  CRA1 forward primer for QRT-PCR
agcgccaggtgcaccgataac

SEQ ID NO: 12 - DNA, 21 bp, Artificial
  CRA 1 reverse primer for QRT-PCR
gttcgcgtttgcgttccactg

SEQ ID NO: 13 - DNA, 23 bp, Artificial
  CRU2 forward primer for QRT-PCR
tctccgccttagcgctcttcgtg

SEQ ID NO: 14 - DNA, 24 bp, Artificial
  CRU2 reverse primer for QRT-PCR
cctgtgcgttttcgtttgtcttga

SEQ ID NO: 15 - DNA, 24 bp, Artificial
  CRU3 forward primer for QRT-PCR
cgacatcgccaactaccaaaacca

SEQ ID NO: 16 - DNA, 21 bp, Artificial
  CRU3 reverse primer for QRT-PCR
cgtcgaacccgctccacaagt

SEQ ID NO: 17 - DNA, 24 bp, Artificial
  2SSSP1 forward primer for QRT-PCR
atctaccgcaccgtcgttgagttc

SEQ ID NO: 18 - DNA, 21 bp, Artificial
  2SSSP1 reverse primer for QRT-PCR
gccttgccttgcttgctggag

SEQ ID NO: 19 - DNA, 24 bp, Artificial
  2SSSP2 forward primer for QRT-PCR
tggcaaacaagctcttcctcgtct

SEQ ID NO: 20 - DNA, 23 bp, Artificial
  2SSSP2 reverse primer for QRT-PCR
gggttgctggcgtcatcttcgtc

SEQ ID NO: 21 - DNA, 22 bp, Artificial
  2SSSP4 forward primer for QRT-PCR
cgcaagcaaatgtggcaaggac

SEQ ID NO: 22 - DNA, 21 bp, Artificial
  2SSSP4 reverse primer for QRT-PCR
aggtggggcaaacgcaaactg

SEQ ID NO: 23 - DNA, 24 bp, Artificial
  ACT2 forward primer for QRT-PCR
gcatgaagatcaaggtggttgcac

SEQ ID NO: 24 - DNA, 25 bp, Artificial
  ACT2 reverse primer for QRT-PCR
atggacctgactcatcgtactcact

SEQ ID NO: 25 - DNA, 4040 bp, Arabidopsis thaliana
caaaagtttgaagctttccttcatgggtttctgatttgtctactaaagttttgatttttttaatttgggtattaataaaaaaagtggggagtgacacaagaagcagtatagtcattagttttccactaagcaaagcacccaaaagttcaccaacaacaaaggcctaatatgtgtctttgtttatgatttgggatcatctgaaacaagatgcgaggagatagcttctcaatgagtattgagaatcttcctgattctccaatgggttcgaggaagaagaagatgcagatcaggaaggtgttcgataaaatgactgaatgggttaccccttggagaagtaatttggagagtccaagagagatgatgattttgcgtggagatgttgaacaagatgagtttcaatacgctagtagtcactgtttgtcttcttactacagtgtctttgttgttcgcctcgctataatggttatgttagcaattttgatagggcttttaacggttctaacatggcatttcacaaggatttatacaaagcagtcgcttcaaacattagcgtatggccttagatacgagcttcttcagcgtccagtcttacggatgtggagcgttttaaacaccacatcagagctaacaacagctcaagtcaagttatctgaatatgtcatcaaaaaatatgacaagccaacaactcaagaagaacttgtggagatgtatcaagcaatgaaggatgtgacatgggctctgtttgcgagtgccaaagctctaaatgccataaccataaactacagaaatggatttgtccaagcttttcacagagatccagcaagtagcagcacattctacatcttctccgatctaaaaaactattccatcagtggaacagggcctgaagatgtaagtgggtggaacaacaagtcaatacacgggaacatgtctgccatttggtatcagcaacagcttgatccagtaacaggggaaaatttgggaaagcctcttaaaattccaccggatgatctgatcaacatcgcaggaatctcacaagtgcctgatggtgaagcttcttggcatgtgacagtgagcaaatacatggactctccgcttctctcagcggctttgcctgtctttgatgcatcaaacaagagtattgtggctgttgtgggggtgacaactgcactttacagcgtaggacagttgatgagagatcttgtagaagtccacggtggacatatttatttgacatctcaagaaggatacttgcttgctacctccacagatggtcctcttttgaaaaatacatcaaacgggcctcagctaatgaaagccactgattcagaagagtgggttataaagtcaggagctcagtggttagagaaaacttatggaagtaagcgccctcacgttgttcatgctgagaatgtaaagcttggtgaccaacgatattacattgattcattctatcttaatctcaagagacttccaattgtaggtgttgtcattattcccaggaagttcattatgggaaaagtggatgaaagggccttcaagactttgattattctgatttctgcttctgtctgcatcttctttattggatgtgtctgcattttaatactcacaaatggagtttcaaaggagatgaagctaagagcagaactgataaggcaactggatgcaagaagaagagctgaagcttcaagcaactacaaaagccagtttttggcaaatatgagtcatgagttgaggacaccaatggctgctgtaattggattactagatattcttatatcggatgattgtctttcaaatgagcaatacgcaacagtcactcagatcagaaagtgctctacagctctccttcggcttctcaataatatactggacttgagcaaggttgagtcaggaaagcttgttctggaagaagctgagtttgatttggggcgagaacttgaaggacttgttgacatgttctcggtgcagtgtattaatcacaatgtagagacagttctagacctctctgatgatatgccagcattagtcagaggagactcagcaagacttgttcaaatatttgcaaatcttataagcaattctataaagtttacaacaacgggtcacattattcttcgtggatggtgcgagaacataaattctctacatgatgagatgagcgtatctgttgacagaaggaaaccatgggctccaatgaagacaaaacaggtgcaacacagaaatcacttgcagaagtcttgtaagaacgcgaacaaaatggttctttggtttgaggttgatgatactggatgtggaatagatccaagcaaatgggactctgtttttgagagctttgagcaagctgatccttctaccactcggacgcacggaggaactggacttggactatgtattgtgcgaaacttggttaacaaaatgggtggagaaatcaaagtagtgcagaagaatggcttagggactcttatgagactgtacttgattttgagtactcctgacactgtagatcaaaacatccagccagatttctccaaatacggtctcgtggttatgctttccatgtatggaagtacagcaagaatgatcacatcaaagtggttgcgaaaacacggcattgcaactgttgaagcatcagactggaacgagctgactcagatcattagagacctcttggagacgggaagccgtgacaacagctttgattcacagcacaatatatctgatccactgagagcagagctttcaaatatagtagaaatcaagaaccctgtgtttgtcatagtggtggatatcggagtacttgatctcaccacaaatatatggaaggaacagcttaactacctcgacagattctccaacaaagcaaagtttgcttggctactgaagcatgacacctccaatacagttaaaacagaacttagacgaaaagggcatgtcatgatggttaacaaaccgttgtacaaggccaaaatgatccagattctggaggctgtaataaaaaaccggaagagaggcttgtgtaatgacttaagaaacagaggaaacggaagtgatgaatcccacgattgcttagaaattgatccaacacaattcgacacttgcagttccgatgattcctcggagacatctggcgagaaacaagtagataaatctgttaaaccaagcaccttgcactctccagtgcttaagaactatctcatcgatgccactaccagcaatgatgactcaacatctgcaagtatgactcaaaagaacccagaagaagaagattggaaggatcggctgtattcaggaattgcgttggatggaaagaatcagaaatctcttgaaggtataaggttcttgctggcagaggatacacccgttcttcaacgagtagccaccataatgcttgagaaaatgggagcaactgtaactgcagtctgggatggacaacaagcagttgattccctcaactacaagtctatcaatgctcaagctccaactgaagaacacaaatcgttcgaagaagaaaccgctaacaaagttacaactcgggagactagtttacggaattcctcgccttatgacttgatcctcatggactgccagatgccaaaaatggatggatatgaggcaacaaaggcgataaggagagcagagattggcacagagctgcacattccaatcgtggcgttgacagcgcatgcaatgtcttctgatgaagcgaaatgcttggaggttggaatggatgcttatctcacaaagcctattgatcgaaagcttatggtctctaccattttgtcactcactaaaccatcagcattccaaacttcattgtccgcttgaagaatcgggtaaaaatctaacaagtgaacttttttagtaggttcaggatcttgcattttgtatatatgtgaatttgtagttgacgatgaggtttttgggggtaaagaaaatcctgttttttggtcattttttgtttactgcaatacgaagaatgttttgtacttaaacattgtatacttgttttaaagatgtgaagaagttaaacgacc

SEQ ID NO: 26 - protein, 1207 aa, Arabidopsis thaliana
MRGDSFSMSIENLPDSPMGSRKKKMQIRKVFDKMTEWVTPWRSNLESPREMMILRGDVEQDEFQYASSHCLSSYYSVFVVRLAIMVMLAILIGLLTVLTWHFTRIYTKQSLQTLAYGLRYELLQRPVLRMWSVLNTTSELTTAQVKLSEYVIKKYDKPTTQEELVEMYQAMKDVTWALFASAKALNAITINYRNGFVQAFHRDPASSSTFYIFSDLKNYSISGTGPEDVSGWNNKSIHGNMSAIWYQQQLDPVTGENLGKPLKIPPDDLINIAGISQVPDGEASWHVTVSKYMDSPLLSAALPVFDASNKSIVAVVGVTTALYSVGQLMRDLVEVHGGHIYLTSQEGYLLATSTDGPLLKNTSNGPQLMKATDSEEWVIKSGAQWLEKTYGSKRPHVVHAENVKLGDQRYYIDSFYLNLKRLPIVGVVIIPRKFIMGKVDERAFKTLIILISASVCIFFIGCVCILILTNGVSKEMKLRAELIRQLDARRRAEASSNYKSQFLANMSHELRTPMAAVIGLLDILISDDCLSNEQYATVTQIRKCSTALLRLLNNILDLSKVESGKLVLEEAEFDLGRELEGLVDMFSVQCINHNVETVLDLSDDMPALVRGDSARLVQIFANLISNSIKFTTTGHIILRGWCENINSLHDEMSVSVDRRKPWAPMKTKQVQHRNHLQKSCKNANKMVLWFEVDDTGCGIDPSKWDSVFESFEQADPSTTRTHGGTGLGLCIVRNLVNKMGGEIKVVQKNGLGTLMRLYLILSTPDTVDQNIQPDFSKYGLVVMLSMYGSTARMITSKWLRKHGIATVEASDWNELTQIIRDLLETGSRDNSFDSQHNISDPLRAELSNIVEIKNPVFVIVVDIGVLDLTTNIWKEQLNYLDRFSNKAKFAWLLKHDTSNTVKTELRRKGHVMMVNKPLYKAKMIQILEAVIKNRKRGLCNDLRNRGNGSDESHDCLEIDPTQFDTCSSDDSSETSGEKQVDKSVKPSTLHSPVLKNYLIDATTSNDDSTSASMTQKNPEEEDWKDRLYSGIALDGKNQKSLEGIRFLLAEDTPVLQRVATIMLEKMGATVTAVWDGQQAVDSLNYKSINAQAPTEEHKSFEEETANKVTTRETSLRNSSPYDLILMDCQMPKMDGYEATKAIRRAEIGTELHIPIVALTAHAMSSDEAKCLEVGMDAYLTKPIDRKLMVSTILSLTKPSAFQTSLSA

(19) United States
(12) Patent Application Publication
(10) Pub. No.: US 2008/0178346 A1
(43) Pub. Date: Jul. 24, 2008

(54) USE OF A GENE ENCODING A HISTIDINE PROTEIN KINASE TO CREATE DROUGHT RESISTANT PLANTS

(76) Inventors: Michael R. Sussman, Cross Plains, WI (US); Dana J. Wohlbach, Sun Prairie, WI (US)

Correspondence Address: BRINKS HOFER GILSON & LIONE, P.O. BOX 10395, CHICAGO, IL, 60610, US

(21) Appl. No.: 11/872,247

(22) Filed: Oct. 15, 2007

Related U.S. Application Data
(60) Provisional application No. 60/851,662, filed on Oct. 13, 2006.

(51) Int. Cl. C12N 15/29; C12N 5/10; A01H 5/00

(52) U.S. Cl. 800/278; 536/23.6; 435/419; 800/298; 800/306

(57) ABSTRACT

Plant expression vectors that include promoter sequences operably linked to heterologous ATHK1 polynucleotides, or complements thereof, encoding polypeptides at least 95% identical to SEQ ID NO:26, where the polynucleotides encode polypeptides that confers drought resistance in the plants. Also provided are transgenic plants with increased drought resistance, methods for creating such plants, overexpressors, and underexpressors of ATHK1. Methods for enhancing drought resistance in plants are also provided.

### CROSS-REFERENCE TO RELATED APPLICATIONS

The present patent document claims the benefit of the filing date under 35 U.S.C. §119(e) of Provisional U.S. Patent Application Ser. No. 60/851,662, filed Oct. 13, 2006, which is hereby incorporated by reference.

### GOVERNMENT INTERESTS

This invention was made with United States government support awarded by the following agencies: DOE, Grant No. DE-FG02-88ER13938; NSF, Grant No. 0116945; and USDA/CSREES, Grant No. 07-CRHF-0-6055. The United States may have certain rights in this invention.

### FIELD OF THE INVENTION

This invention relates generally to the fields of plant physiology, genetics, and molecular biology. In particular, this invention provides genes and proteins that confer drought resistance, drought resistant plants, and methods of enhancing drought resistance in plants.

### BACKGROUND

To cope with drought or salinity stress, plants must be able to sense, rapidly respond, and adapt to osmotic changes. A major response to these stresses occurs at the gene expression level, resulting in increased production of osmolytes, antioxidants, and other metabolites to increase drought stress tolerance and/or resistance. Some of the genes involved in this response have been identified, however definitive evidence for a plant osmotic stress receptor has been lacking.

Osmotic changes are most often thought of in the context of the environmental conditions in which a plant is growing. However, osmotic changes also occur during seed formation. In order to form a viable seed, a plant must establish a proper osmotic balance by successfully coordinating many cellular events. Seeds gradually lose their viability over time. The exact mechanisms that lead to a loss of viability are not well established, and the genetic factors affecting seed quality are little understood.

Two-component histidine kinase signal transduction pathways are involved in various responses in many prokaryotes, fungi, and plants. ATHK1, an Arabidopsis (Arabidopsis thaliana L.) histidine kinase, has been shown to complement a deletion mutant of the yeast osmosensing histidine kinase SLN1, and can function as an osmosensor in yeast (Urao et al., 1999, Plant Cell 11: 1743-1754). However, lack of sequence homology in the external domains between ATHK1 and SLN1, and promiscuous cross-talk among histidine kinases, reduces confidence that complementation in yeast reflects receptor function in plants and precludes a clear statement of ATHK1 function. Even though the nucleotide and amino acid sequences of ATHK1 are known (Urao et al., 1999), no genetic evidence has been presented that conclusively establishes the role of ATHK1.

A fundamental challenge of the current understanding on osmotic stress tolerance in plants is the molecular characterization of the components involved in the stress perception, signal transduction, and response. Understanding and regulating increased osmotic stress tolerance should result in agricultural applications as society assumes adaptive, rather than preventive, reaction to increasing drought conditions worldwide. The present invention provides compounds and methods for facilitating this objective.

### BRIEF SUMMARY

This invention provides expression cassettes for plants that include promoter sequences operably linked to heterologous ATHK1 polynucleotides, or complements thereof, encoding polypeptides that are at least 95% identical to SEQ ID NO:26. The polynucleotides encode polypeptides that confer drought resistance in plants. The ATHK1 polynucleotides, or the complements thereof, may be at least 95% identical to SEQ ID NO:25. The ATHK1 polynucleotides, or the complements thereof, may comprise positions from about 208 to about 3831 of SEQ ID NO:25. The promoters may, e.g., be constitutive, tissue-specific, or inducible.

This invention provides plant expression vectors comprising these expression cassettes for plants. This invention provides host cells comprising the expression cassettes for plants. The host cells may be plant cells.

This invention provides transgenic plants comprising isolated polynucleotides that encode: a) polypeptides comprising amino acid sequences that are at least 95% identical to SEQ ID NO:26, or b) polypeptides comprising at least 1147 amino acids of SEQ ID NO:26, where the polypeptides confer drought resistance in the transgenic plants. Also, this invention provides a seed stock from which transgenic plants may be produced or regenerated. The transgenic plants may be dicotyledons, and in particular they may be brassicaceous plants.

This invention provides methods of producing transgenic plants with enhanced drought resistance. The methods include introducing into the plants isolated polynucleotides that encode: a) polypeptides comprising amino acid sequences that are at least 95% identical to SEQ ID NO:26, or b) polypeptides comprising at least 1147 amino acids of SEQ ID NO:26, where the polypeptides confer drought resistance in the transgenic plants.

This invention provides plants with enhanced drought resistance. The plants include recombinant expression cassettes for plants comprising promoters operably linked to polynucleotides encoding polypeptides at least 95% identical to SEQ ID NO:26. The plants may include polynucleotides encoding polypeptides that are at least 95% identical to SEQ ID NO:25. The plants may include promoters that are, e.g., constitutive, tissue-specific, or inducible.

This invention provides methods of producing transgenic plants with enhanced drought resistance. The methods include introducing into the plants recombinant expression cassettes for plants comprising polynucleotides encoding polypeptides that are at least 95% identical to SEQ ID NO:26.

This invention provides methods of growing crops with enhanced drought resistance. The methods include introducing into the crops expression cassettes for plants comprising promoter sequences operably linked to heterologous ATHK1 polynucleotides, or complements thereof, encoding polypeptides that are at least 95% identical to SEQ ID NO:26, where the polynucleotides encode polypeptides that confers drought resistance in the crops. The crops may be dicotyledons, and in particular they may be brassicaceous plants.

This invention provides transgenic plants which overexpress polynucleotides encoding polypeptides at least 95% identical to SEQ ID NO:26 in the plants, as compared to non-transformed plants. The overexpression causes increased drought resistance in the transgenic plants. The transgenic plants may be dicotyledonous plants. The invention also provides seeds obtained by growing these transgenic plants.

This invention provides transgenic plants which underexpress polynucleotides encoding polypeptides at least 95% identical to SEQ ID NO:26 in the plants, as compared to non-transformed plants. The underexpression causes decreased drought resistance in the transgenic plants. The transgenic plants may be dicotyledonous plants. The invention also provides seeds obtained by growing these transgenic plants.

### BRIEF DESCRIPTION OF THE DRAWINGS

FIG. 1 is a schematic diagram of: (a) the ATHK1 protein; (b) locations of athk1 T-DNA inserts, structures of athk1 alleles and the ATHK1 fragment used for molecular complementation.

FIG. 2 shows an image and a graph depicting relative expression levels of ATHK1 RNA in athk1 knockouts, wild-type, ATHK1-rescued athk1 knockouts, and 35S ATHK1 plants.

FIG. 3 shows a graph illustrating the altered osmotic response of ATHK1 alleles during root elongation.

FIG. 4 depicts graphs showing seed phenotypes.

FIG. 5 is an alignment showing the lack of amino acid similarity between ATHK1 and SLN1.

FIG. 6 is a graph showing altered stomatal response of ATHK1 alleles.

FIG. 7 is a graph showing altered osmotic and ionic sensitivity of ATHK1 alleles during germination.

FIG. 8 depicts graphs showing how ATHK1 alleles display altered abscisic acid (ABA) phenotypes.

FIG. 9 shows graphs illustrating altered expression of ABA-regulated seed transcripts in ATHK1 mutants.

FIG. 10 shows graphs illustrating altered expression of ABA biosynthetic genes in ATHK1 mutants.

FIG. 11 shows graphs illustrating altered expression of osmotic stress-responsive genes in ATHK1 mutants and expression of ATHK1 in response to stress.

### DETAILED DESCRIPTION OF THE PRESENTLY PREFERRED EMBODIMENTS

### Definitions

The following definitions are provided in order to aid the reader in understanding the detailed description of the present invention.

The terms “a”, “an”, “the” and the like, unless otherwise indicated, include plural forms.

The term “osmosis” refers to the movement of a solvent (as water) through a semipermeable membrane (as of a living cell) into a solution of higher solute concentration that tends to equalize the concentrations of solute on the two sides of the membrane.

The term “drought” refers to the inadequacy of water availability from an agricultural point of view, including insufficient precipitation and soil moisture-storage capacity, in quantity and distribution during the life cycle of a crop plant, which restricts the expression of the full genetic potential of the plant.

The phrase “drought resistance response” refers to a change in metabolism, biosynthetic activity, or gene expression that enhances a plant's ability to respond to water deficit and/or osmotic stress. Examples of plant drought responses and/or resistance include, but are not limited to, drought escape, drought avoidance, and drought tolerance (Mitra, 2001, Curr. Sci. 80: 758-763). Preferred genes and proteins of the present invention, when expressed in plant, are expected to confer drought resistance or drought tolerance in the plant. The terms “drought resistance” and “drought tolerance” are used interchangeably herein and refer to any indicia of success in the resistance and/or tolerance of drought.

The phrase “useful for conferring drought resistance” refers to the ability to initiate a drought resistance response in a plant and subsequently confer drought resistance in the plant. Transgenic plants of the present invention having enhanced drought resistance have the ability to mount a drought resistance response to drought-causing agents, such as insufficient water availability, presence of osmotic substances, salts, etc.

The terms “drought resistance genes” or “drought resistance proteins” refer to genes or their encoded proteins whose expression or synthesis confers drought resistance.

The term “ATHK1” (Arabidopsis thaliana histidine kinase 1) refers to a histidine kinase isolated from Arabidopsis (Arabidopsis thaliana L.). The nucleotide sequence of ATHK1 (SEQ ID NO:25) and the amino acid sequence of ATHK1 (SEQ ID NO:26) appear in the gene bank databases GDSB, DDBJ, EMBL, and NCBI as accession number AB010914. The nucleotide and deduced amino acid sequences of ATHK1 are described in Urao et al., 1999. For use in the present invention, the term “ATHK1” also refers to polymorphic variants, mutants, alleles, and interspecies homologs of the histidine kinase ATHK1 gene and ATHK1 protein cloned from Arabidopsis thaliana.

The terms “nucleic acid” or “polynucleotide sequence” refer to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

The terms “coding sequence” or “coding region” refer to a nucleic acid molecule having sequence information necessary to produce a gene product, when the sequence is expressed.

As used herein, “genetically modified” or “transgenic” plant cell or plant means a plant cell or plant stably incorporating a nucleic acid construct introduced by transformation methods. The term “wild type” refers to an untransformed plant cell or plant.

The phrases “operably linked” or “operably inserted” mean that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g. enhancers) in an expression cassette.

The term “expression”, as used herein, refers to the transcription and stable accumulation of sense (mRNA) or antisense RNA derived from the nucleic acid fragment of the invention. Expression may also refer to translation of mRNA into a polypeptide. “Overexpression” refers to the production of a gene product in transgenic organisms that exceeds levels of production in normal or non-transformed organisms. “Underexpression” refers to the production of a gene product in transgenic organisms at levels below that of levels of production in normal or non-transformed organisms. “Co-suppression” refers to the production of sense RNA transcripts capable of suppressing the expression of identical or substantially similar foreign or endogenous genes (U.S. Pat. No. 5,231,020, incorporated herein by reference).

“Altered levels” refers to the production of gene product(s) in transgenic organisms in amounts or proportions that differ from that of normal or non-transformed organisms.

“Antisense inhibition” refers to the production of antisense RNA transcripts capable of suppressing the expression of the target protein.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region”, or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “nucleic acid construct” or “DNA construct” is sometimes used to refer to a coding sequence or sequences operably linked to appropriate regulatory sequences and inserted into a expression cassette for transforming a cell. This term may be used interchangeably with the term “transforming DNA” or “transgene”. Such a nucleic acid construct may contain a coding sequence for a gene product of interest, along with a selectable marker gene and/or a reporter gene.

A “heterologous” region of a nucleic acid construct is an identifiable segment (or segments) of the nucleic acid molecule within a larger molecule that is not found in association with the larger molecule in nature. When the heterologous region encodes a plant gene, the gene will usually be flanked by DNA that does not flank the plant genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein. The term “DNA construct” is also used to refer to a heterologous region, particularly one constructed for use in transformation of a cell.

The term “recombinant” when used with reference, e.g., to a cell, or nucleic acid, protein, expression cassette, or vector, indicates that the cell, nucleic acid, protein, expression cassette, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, underexpressed or not expressed at all.

The term “host cell” refers to a cell from any organism. Preferred host cells are derived from plants, bacteria, yeast, fungi, insects, or animals. The term “recombinant host cell” (or simply “host cell”) refers to a cell into which a recombinant expression vector has been introduced. It should be understood that the term “host cell” is intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. Methods for making transformed plants, i.e., transgenic plants or genetically modified plants, are disclosed, e.g., in U.S. Pat. No. 5,723,765, which is incorporated herein by reference.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seed (including embryo, endosperm, and seed coat) and fruit (the mature ovary), plant tissue (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, bryophytes, and multicellular algae. It includes plants of a variety of ploidy levels, including aneuploid, polyploid, diploid, haploid and hemizygous.

A polynucleotide “exogenous to” an individual plant is a polynucleotide which is introduced into the plant, or a predecessor generation of the plant, by any means other than by a sexual cross. Examples of means by which this can be accomplished are described below, and include Agrobacterium-mediated transformation, biolistic methods, electroporation, in planta techniques, and the like.

“Increased or enhanced expression or activity of a polypeptide of the present invention,” or “increased or enhanced expression or activity of a polynucleotide encoding a polypeptide of the present invention,” refers to an augmented change in activity of the polypeptide or protein. Examples of such increased activity or expression include the following: (1) activity of the protein or expression of the gene encoding the protein is increased above the level of that in wild-type, non-transgenic control plants; (2) activity of the protein or expression of the gene encoding the protein is in an organ, tissue or cell where it is not normally detected in wild-type, non-transgenic control plants (i.e., spatial distribution of the protein or expression of the gene encoding the protein is altered); (3) activity of the protein or expression of the gene encoding the protein is increased when activity of the protein or expression of the gene encoding the protein is present in an organ, tissue or cell for a longer period than in a wild-type, non-transgenic controls (i.e., duration of activity of the protein or expression of the gene encoding the protein is increased).

The phrase “decreased expression or activity of a protein or polypeptide of the present invention,” or “decreased expression or activity of a nucleic acid or polynucleotide encoding a protein of the present invention,” refers to a decrease in activity of the protein. An example of such decreased activity or expression includes the decrease in activity of the protein or expression of the gene encoding the protein below the level of that in wild-type, non-transgenic control plants.

The term “expression cassette” refers to a nucleic acid construct, which when introduced into a host cell, results in transcription and/or translation of a RNA or polypeptide, respectively. Expression cassettes can be derived from a variety of sources depending on the host cell to be used for expression. For example, an expression cassette can contain components derived from a viral, bacterial, insect, plant, or mammalian source. In the case of both expression of transgenes and inhibition of endogenous genes (e.g., by antisense, or sense suppression) one of skill will recognize that the inserted polynucleotide sequence need not be identical and can be “substantially identical” to a sequence of the gene from which it was derived.

The terms “expression cassette for plants” or “plant expression cassette” refer to an expression cassette that can be used to transform plants. Examples of expression cassettes for plants can be found in PCT Patent Publications Nos. WO/1990/002189 and WO/2000/026388; Ainley and Key, 1990, Plant Mol. Biol. 14: 949-967; Birch, 1997, Annu. Rev. Plant Physiol. Plant Mol. Biol. 48: 297-326.

The term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. The terms “expression vector for plants” or “plant expression vector” refer to an expression vector that is suitable for nucleic acid expression in a plant cell.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or proteins for which antisera or monoclonal antibodies are available.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

The terms “isolated”, “purified”, or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

In the case of both expression of transgenes and inhibition of endogenous genes (e.g., by antisense, or sense suppression) one of skill will recognize that the inserted polynucleotide sequence need not be identical and may be substantially identical to a sequence of the gene from which it was derived. These variants are specifically covered by this term.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “polynucleotide sequence from” a particular gene. In addition, the term specifically includes sequences (e.g., full length sequences) substantially identical with a gene sequence encoding a protein of the present invention and that encode proteins or functional fragments that retain the function of a protein of the present invention, i.e. enhanced resistance to drought.

In the case of polynucleotides used to inhibit expression of an endogenous gene, the introduced sequence need not be perfectly identical to a sequence of the target endogenous gene. The introduced polynucleotide sequence will typically be at least substantially identical to the target endogenous sequence.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by methods commonly known in the art, e.g., the local homology algorithm (Smith and Waterman, 1981, Adv. Appl. Math. 2: 482-489), by the search for similarity method (Pearson and Lipman, 1988, Proc. Natl. Acad. Sci. USA 85: 2444-2448), by computerized implementations of these algorithms (GAP, BESTFIT, BLAST, FASTA, and TFASTA in the Wisconsin Genetics Software Package, Genetics Computer Group (GCG), Madison, Wis.), or by inspection.

In a particularly preferred embodiment, protein and nucleic acid sequence identities are evaluated using the Basic Local Alignment Search Tool (“BLAST”) which is well known in the art (Karlin and Altschul, 1990, Proc. Natl. Acad. Sci. USA, 87: 2267-2268; Altschul et al., 1997, Nucl. Acids Res. 25: 3389-3402). The BLAST programs can be used with the default parameters or with modified parameters provided by the user.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 98.7%, or 99%. Polypeptides that are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine. Accordingly, polynucleotides of the present invention encoding a protein of the present invention include nucleic acid sequences that have substantial identity to the nucleic acid sequence of ATHK1. Polypeptides or proteins of the present invention include amino acid sequences that have substantial identity to the amino acid sequence of ATHK1.

This invention also relates to nucleic acids that selectively hybridize to the exemplified sequences, including hybridizing to the exact complements of these sequences. The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments. DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach is to vary the temperature: higher relative temperatures result in more stringent reaction conditions. See Ausubel et al., 1993, Current Protocols in Molecular Biology, Volumes 1-3, John Wiley & Sons, Inc., for an excellent explanation of the stringency of hybridization reactions.

General Overview

Generally, the nomenclature and the laboratory procedures in recombinant DNA technology described below are those well known and commonly employed in the art. Standard techniques are used for cloning, DNA and RNA isolation, amplification and purification. Enzymatic reactions involving DNA ligase, DNA polymerase, restriction endonucleases and the like are generally performed according to the manufacturer's specifications. These techniques and various other techniques are generally performed according to Sambrook et al., 1989, Molecular Cloning—A Laboratory Manual, Cold Spring Harbor Laboratory, Cold Spring Harbor, N.Y.; Ausubel et al., 1993, Current Protocols in Molecular Biology, Volumes 1-3, John Wiley & Sons, Inc.; and Kriegler, 1990, Gene Transfer and Expression: A Laboratory Manual, Stockton Press, New York, each of which is incorporated herein by reference in its entirety. Enzymatic reactions involving DNA ligase, DNA polymerase, restriction endonucleases and the like, are generally performed according to the manufacturer's specifications.

Polynucleotides and polypeptides that confer drought resistance

The role of ATHK1 the regulation of signal transduction of osmotic stress in Arabidopsis thaliana was suggested by Hao et al., 2004, Zhi Wu Sheng Li Yu Fen Zi Sheng Wu Xue Xue Bao 30: 553-560. As described in the present invention, it has been discovered that engineered overexpression of ATHK1 in plants results in enhanced drought tolerance. Thus, the preferred polynucleotide of the present invention encodes a polypeptide useful for conferring drought resistance a plant. Polynucleotides of the present invention can be identified by their ability to hybridize under low stringency conditions to nucleic acid probes having the sequence of ATHK1 or the complement thereof, and fragments thereof.

A preferred “ATHK1 polypeptide” or “ATHK1 protein” of the present invention is expected to confer drought resistance in a plant, and has one or more of the following: (I) substantial identity to the amino acid sequence of ATHK1 as described in Urao et al., 1999; (ii) includes one or more of the following domains or motifs: TMD, transmembrane domain; HisKA, histidine kinase domain; HATPase, ATP binding domain; REC, receiver domain; (iii) binds to antibodies raised against an immunogen comprising an amino acid sequence of ATHK1. Polypeptides of the present invention include polymorphic variants, mutants, and interspecies homologs of ATHK1. Polypeptides of the present invention also include functional equivalents or fragments of ATHK1. A functional fragment or functional equivalent or functional homolog of a polypeptide of the present invention is a polypeptide that is homologous to the specified polypeptide but has one or more amino acid differences from the specified polypeptide. A functional fragment or equivalent of a polypeptide retains at least some, if not all, of the activity of the specified polypeptide.

In general, an ATHK1 polypeptide functional homolog that preserves ATHK1 polypeptide-like function includes any homolog in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the amino acid substitution is a conservative substitution. Conservative substitutions whereby an amino acid of one class is replaced with another amino acid of the same type fall within the scope of the invention so long as the substitution does not materially alter the biological activity of the compound. For example, a functional equivalent of ATHK1 shares the same amino acid sequence as ATHK1 except for a few amino acid differences, e.g., substitutions, insertions, or deletions. When expressed in a plant, both ATHK1 and its functional homolog are expected to confer drought resistance.

The present invention also provides expression regulatory elements, and in particular a native promoter or variant of a native promoter from Arabidopsis that can be used to express ATHK1 genes and proteins of the present invention in a variety of plants.

The isolation of sequences from the genes used in the methods of the present invention may be accomplished by a number of techniques. For instance, oligonucleotide probes based on the sequences disclosed here can be used to identify the desired gene in a cDNA or genomic DNA library from a desired plant species. To construct genomic libraries, large segments of genomic DNA are generated by random fragmentation, e.g. using restriction endonucleases, and are ligated with vector DNA to form concatemers that can be packaged into the appropriate vector. The cDNA or genomic library can then be screened using a probe based upon the sequence of a cloned gene such as the polynucleotides disclosed here. Probes may be used to hybridize with genomic DNA or cDNA sequences to isolate homologous genes in the same or different plant species.

Alternatively, the nucleic acids of interest can be amplified from nucleic acid samples using amplification techniques. For instance, polymerase chain reaction (PCR) technology can be used to amplify sequences directly from mRNA, from cDNA, from genomic libraries or cDNA libraries. PCR and other in vitro amplification methods may also be useful to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying genes encoding a protein of the present invention from plant tissues are generated from comparisons of the sequences provided herein. For a general overview of PCR see PCR Protocols, 2003, Bartlett and Stirling, eds., 2^{nd} edition, Humana Press, Totowa, N.J. For examples of primers used see examples section below.

Polynucleotides may also be synthesized by well-known techniques as described in the technical literature (Adams et al., 1983, J. Am. Chem. Soc. 105: 661-663). Double stranded DNA fragments may then be obtained either by synthesizing the complementary strand and annealing the strands together under appropriate conditions, or by adding the complementary strand using DNA polymerase with an appropriate primer sequence.

Once a nucleic acid is isolated, standard methods can be used to determine if the nucleic acid is a preferred nucleic acid of the present invention and therefore encodes a preferred protein of the present invention, e.g., by using structural and functional assays known in the art. For example, the skilled practitioner can compare the sequence of a putative nucleic acid sequence thought to encode a preferred protein of the present invention to a nucleic acid sequence encoding a preferred protein of the present invention to determine if the putative nucleic acid is a preferred polynucleotide of the present invention.

One useful method to produce the nucleic acids of the present invention is to isolate and, if desired, modify the nucleic acid sequences of the present invention. Methods of sequence-specific mutagenesis of a nucleic acid are known. In addition, Ausubel et al., 1993, describe oligonucleotide-directed mutagenesis as well as directed mutagenesis of nucleic acids using PCR. Such methods are useful to insert specific codon changes in the nucleic acids of the invention.

One of skill will recognize that the polypeptides encoded by the genes of the invention, like other proteins, have different domains which perform different functions. Thus, the gene sequences need not be full length, so long as the desired functional domain of the protein is expressed. As well, the expressed proteins may be fusion proteins, labeled proteins, etc.

Preparation of Recombinant Vectors

The present invention further provides recombinant expression cassettes and expression vectors that include a nucleic acid encoding an ATHK1 polypeptide which, when produced in a plant, confers drought resistance in the plant.

Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both (e.g., shuttle vectors), and selection markers for both prokaryotic and eukaryotic systems. Vectors are suitable for replication and integration in prokaryotes, eukaryotes, or preferably both. A catalogue of bacteria and bacteriophages useful for cloning is provided, e.g., by the American Type Culture Collection (ATCC), Manassas, Va. Additional basic procedures for sequencing, cloning and other aspects of molecular biology and underlying theoretical considerations are also found in Watson et al., 1992, Recombinant DNA, Second Ed., Scientific American Books, NY.

To use isolated sequences in the above techniques, recombinant DNA vectors suitable for transformation of plant cells are prepared. Techniques for transforming a wide variety of higher plant species are well known and described in the scientific literature (Weising et al., 1988, Annu. Rev. Genet. 22: 421-477; Chrispeels et al., 2003, Plants, Genes, and Crop Biotechnology, Second Ed., James and Bartlett; Regulation of Gene Expression in Plants: The Role of Transcript Structure and Processing, Bassett, ed., Springer, New York, 2007). A DNA sequence coding for the desired polypeptide, for example a cDNA sequence encoding a full length protein, will preferably be combined with transcriptional and translational initiation regulatory sequences that will direct the transcription of the sequence from the gene in the intended tissues of the transformed plant.

Alternatively, the plant promoter may direct expression of the polynucleotide of the invention in a specific tissue (tissue-specific promoters), organ (organ-specific promoters), or may be otherwise under more precise environmental control (inducible promoters). Examples of tissue-specific promoters under developmental control include promoters that initiate transcription only in certain tissues, such as fruit, seeds, flowers, pistils, or anthers. Suitable promoters include those from genes encoding storage proteins or the lipid body membrane protein, oleosin. Examples of environmental conditions that may affect transcription by inducible promoters include anaerobic conditions, elevated temperature, or the presence of light.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Nucleic acid sequences of the present invention can be expressed recombinantly in plant cells to enhance and increase levels of endogenous plant transcription factors. A variety of different expression constructs, such as expression cassettes and vectors suitable for transformation of plant cells can be prepared. A DNA sequence coding for a polypeptide described in the present invention can be combined, for example, with cis-acting (promoter and enhancer) transcriptional regulatory sequences to direct the timing, tissue type and levels of transcription in the intended tissues of the transformed plant. Translational control elements can also be used.

The invention provides a nucleic acid operably linked to a promoter which, in some embodiments, is capable of driving the transcription of the coding sequence in plants. The promoter can be, e.g., derived from plant or viral sources. The promoter can be, e.g., constitutively active, inducible, or tissue specific. In construction of recombinant expression cassettes, vectors, transgenics, of the invention, different promoters can be chosen and employed to differentially direct gene expression, e.g., in some or all tissues of a plant or animal. Typically, desired promoters are identified by analyzing the 5′ sequences of a genomic clone corresponding to the genes described.

In one embodiment, a promoter fragment can be employed which will direct expression of a nucleic acid of the present invention in all transformed cells or tissues, e.g. as those of a regenerated plant. Such promoters are referred to herein as “constitutive” promoters and are active under most environmental conditions and states of development or cell differentiation. Examples of constitutive promoters include those from viruses which infect plants, such as the cauliflower mosaic virus (CaMV) 35S transcription initiation region (Dagless et al., 1997, Arch. Virol. 142: 183-191); the 1′- or 2′-promoter derived from T-DNA of Agrobacterium tumefaciens (O'Grady and Gurley, 1995, Plant Mol. Biol. 29: 99-108); the promoter of the tobacco mosaic virus; the promoter of Figwort mosaic virus (Maiti et al., 1997, Transgenic Res. 6: 143-156); actin promoters, such as the Arabidopsis actin gene promoter (Huang et al., 1997, Plant Mol. Biol. 33: 125-139); alcohol dehydrogenase (Adh) gene promoters (Millar and Dennis, 1996, Plant Mol. Biol. 31: 897-904); ACT11 from Arabidopsis (Huang et al., 1996, Plant Mol. Biol. 33: 125-139), Cat3 from Arabidopsis (Zhong et al., 1996, Mol. Gen. Genet. 251:196-203), the gene encoding stearoyl-acyl carrier protein desaturase from Brassica napus (Solocombe et al., 1994, Plant Physiol. 104: 1167-1176), gpc1 from maize (Martinez et al., 1989, J. Mol. Biol. 208: 551-565), gpc2 from maize (Manjunath et al., 1997, Plant Mol. Biol. 33: 97-112), other transcription initiation regions from various plant genes known to those of skill (Holtorf et al., 1995, Plant Mol. Biol. 29: 637-646).

Alternatively, a plant promoter can direct expression of the nucleic acids described in the present invention under the influence of changing conditions, e.g., changing environmental conditions. Examples of environmental conditions that may effect transcription by inducible promoters include anaerobic conditions, elevated temperature, drought, or the presence of light. Examples of developmental conditions that may affect transcription by inducible promoters include senescence and embryogenesis. Such promoters are referred to herein as “inducible” promoters. For example, the invention incorporates the drought-inducible promoter of maize (Busk et al., 1997, Plant J. 11: 1285-1295); the cold, drought, and high salt inducible promoter from potato (Kirch et al., 1997, Plant Mol. Biol. 33: 897-909). The invention incorporates senescence inducible promoters, such as SAG 12 (Gan and Amasino, 1995, Science 270: 1986-1988) and the embryogenesis related promoters of LEC1 (Lotan et al., 1998, Cell 93: 1195-1205), LEC2 (Stone et al., 2001, Proc. Natl. Acad. Sci. USA 98: 11806-11811), FUS3 (Luerssen et al., 1998, Plant J. 15: 755-764), AtSERK1 (Hecht et al., 2001, Plant Physiol. 127: 803-816), AGL15 (Heck et al., 1995, Plant Cell 7: 1271-1282), and BBM (BABYBOOM) (Boutilier et al., 2002, Plant Cell 14:1737-1749).

Alternatively, plant promoters which are inducible upon exposure to plant hormones, such as auxins or cytokinins, can be used to express the nucleic acids of the invention. For example, the invention can use the auxin response elements E1 promoter fragment (AuxREs) in the soybean (Glycine max L.) (Liu et al., 1997, Plant Physiol. 115: 397-407); the auxin-responsive Arabidopsis GST6 promoter (also responsive to salicylic acid and hydrogen peroxide) (Chen et al., 1996, Plant J. 10: 955-966); the auxin-inducible parC promoter from tobacco (Sakai et al., 1996, Plant Cell Physiol. 37: 906-913); a plant biotin response element (Streit and Phillips, 1997, Mol. Plant. Microbe Interact. 10: 933-937); and the promoter responsive to the stress hormone abscisic acid (Sheen, 1996, Science 274: 1900-1902). The invention can also use the cytokinin inducible promoters of ARR5 and ARR6 (Brandstatter and Kieber, 1998, Plant Cell 10: 1009-1019), ARR2 (Hwang and Sheen, 2001, Nature 413: 383-389), the ethylene responsive promoter of ERF1 (Solano et al., 1998, Genes Dev. 12: 3703-3714), and the β-estradiol inducible promoter of XVE (Zuo et al., 2000, Plant J. 24: 265-273).

Plant promoters which are inducible upon exposure to chemical reagents that can be applied to the plant, such as herbicides or antibiotics, are also used to express the nucleic acids of the invention. For example, the maize In2-2 promoter, activated by benzenesulfonamide herbicide safeners, can be used (De Veyider et al., 1997, Plant Cell Physiol. 38: 568-577) as well as the promoter of the glucocorticoid receptor protein fusion inducible by dexamethasone application (Aoyama and Chua, 1997, Plant J. 11: 605-612); application of different herbicide safeners induces distinct gene expression patterns, including expression in the root, hydathodes, and the shoot apical meristem. The coding sequence of the described nucleic acids can also be under the control of, e.g., a tetracycline inducible promoter, e.g., as described with transgenic tobacco plants containing the Avena sativa L. (oat) arginine decarboxylase gene (Masgrau et al., 1997, Plant J. 11: 465-473); or a salicylic acid responsive element (Stange et al., 1997, Plant J. 11: 1315-1324).

Alternatively, inducible promoters include the tetracycline repressor/operator controlled promoter, the heat shock gene promoters, stress (e.g., wounding)-inducible promoters, defense responsive gene promoters (e.g. phenylalanine ammonia lyase genes), wound induced gene promoters (e.g. hydroxyproline rich cell wall protein genes), chemically-inducible gene promoters (e.g., nitrate reductase genes, glucanase genes, chitinase genes, etc.) and dark-inducible gene promoters (e.g., asparagine synthetase gene). Pathogen-inducible and wound-inducible promoters include, but are not limited to, promoters of genes encoding lipoxygenases (Peng et al., 1994, J. Biol. Chem. 269: 3755-3761); promoters of genes encoding peroxidases (Chittoor et al., 1997, Mol. Plant-Microbe Interactions 10: 861-871); promoters of genes encoding hydroxymethylglutaryl-CoA reductase (Nelson et al., 1994, Plant Mol. Biol. 25: 401-412); promoters of genes encoding phenylalanine ammonia lyase (Ozeki et al., 2003, J. Plant Research 116: 155-159); promoters of genes encoding glutathione-S-transferase; promoters from pollen-specific genes, such as corn Zmg13; promoters from genes encoding chitinases (Zhu and Lamb, 1991, Mol. Gen. Genet. 226: 289-296); promoters from plant viral genes, either contained on a bacterial plasmid or on a plant viral vector (Hammond-Kosack et al., 1994, Mol. Plant-Microbe Interactions 8: 181-185); promoters from genes involved in the plant respiratory burst (Groom et al., 1996, Plant J. 10: 515-522); and promoters from plant anthocyanin pathway genes (Gandikota et al., 2004, Mol. Breeding. 7: 73-83).

Alternatively, the plant promoter can direct expression of the polynucleotide of the invention in a specific tissue. Such promoters are referred to herein as “tissue-specific promoters”. The tissue-specific promoters are transcriptional control elements that are only active in particular cells or tissues at specific times during plant development, such as in vegetative tissues or reproductive tissues.

Examples of tissue-specific promoters under developmental control include promoters that initiate transcription only (or primarily only) in certain tissues, such as vegetative tissues, e.g., roots, leaves or stems, or reproductive tissues, such as fruit, ovules, seeds, pollen, pistils, flowers, or any embryonic tissue. Reproductive tissue-specific promoters can be, e.g., ovule-specific, embryo-specific, endosperm-specific, integument-specific, seed and seed coat-specific, pollen-specific, petal-specific, sepal-specific, or some combination thereof.

Suitable seed-specific promoters can be derived from the following genes: MAC1 from maize (Sheridan et al., 1996, Genetics 142: 1009-1020); Cat3 from maize (Abler and Scandalios, 1993, Plant Mol. Biol. 22: 1031-1038); viviparous-1 from Arabidopsis (Suzuki et al., 2003, Plant Physiol. 132: 1664-1667); atmyc1 from Arabidopsis (Urao et al., 1996, Plant Mol. Biol. 32: 571-576; Conceicao et al., 1994, Plant J. 5: 493-505); napA and BnCysP1 from Brassica napus (Josefsson et al., 1987, J. Biol. Chem. 26: 12196-1301; Wan et al., 2002, Plant J. 30:1-10); and the napin gene family from Brassica napus (Sjodahl et al., 1995, Planta 197: 264-271). Fruit-specific promoters include the promoter from the CYP78A9 gene (Ito and Meyerowitz, 2000, Plant Cell 12: 1541-1550).

The promoter of the ovule-specific BEL1 gene described in Reiser et al., 1995, Cell 83: 735-742, GenBank No. U39944, can also be used. The egg and central cell specific FIE1 promoter is also a useful reproductive tissue-specific promoter.

Sepal and petal specific promoters can also used be to express nucleic acids encoding a B3 domain protein in a reproductive tissue-specific manner. For example, the Arabidopsis floral homeotic gene APETALA1 (AP1) encodes a putative transcription factor that is expressed in young flower primordia, and later becomes localized to sepals and petals (Gustafson-Brown et al., 1994, Cell 76: 131-143; Mandel et al., 1992, Nature 360: 273-277). A related promoter, for AP2, a floral homeotic gene that is necessary for the normal development of sepals and petals in floral whorls, is also useful (Drews et al., 1991, Cell 65: 991-1002; Bowman et al., 1991, Plant Cell 3: 749-758). Another useful promoter is that controlling the expression of the unusual floral organs (ufo) gene of Arabidopsis, whose expression is restricted to the junction between sepal and petal primordia (Bossinger and Smyth, 1996, Development 122: 1093-1102). A pollen-specific promoter that has been identified in maize (Guerrero et al., 1990, Mol. Gen. Genet. 224: 161-168) can also be used.

Promoters specific for pistil and silique valves, inflorescence meristems, cauline leaves, and the vasculature of stem and floral pedicels include promoters from the FUL gene (Mandel and Yanofsky, 1995, Plant Cell 7: 1763-1771). Promoters specific for developing carpels, placenta, septum, and ovules may also used to express nucleic acids of the present invention in a tissue-specific manner. They include promoters from the SHP1 and SHP2 genes (Flanagan et al., 1996, Plant J. 10: 343-353). The pistil specific promoter in the potato (Solanum tuberosum) SK2 gene, encoding a pistil specific basic endochitinase (Ficker et al., 1997, Plant Mol. Biol. 35: 425-431), can also be used.

Other suitable promoters include those from genes encoding embryonic storage proteins. For example, the gene encoding the 2S storage protein from Brassica napus (Dasgupta, 1993, Gene 133: 301-302); the 2s seed storage protein gene family from Arabidopsis; the gene encoding oleosin from Brassica napus (GenBank No. M63985); the genes encoding oleosin A (Genbank No. U09118) and oleosin B (Genbank No. U09119) from soybean; the gene encoding oleosin from Arabidopsis (GenBank No. Z17657); the gene encoding oleosin from maize (Lee and Huang, 1994, Plant Mol. Biol. 26: 1981-1987); and the gene encoding low molecular weight sulfur rich protein from soybean (Choi et al., 1995, Mol Gen, Genet. 246: 266-268), can be used. The tissue-specific E8 promoter from tomato is particularly useful for directing gene expression so that a desired gene product is located in fruits. Suitable promoters may also include those from genes expressed in vascular tissue, such as the ATHB-8, AtPIN1, AtP5K1 or TED3 genes (Baima et al., 2001, Plant Physiol. 126: 643-655; Galaweiler et al., 1998, Science 282: 2226-2230; Elge et al., 2001, Plant J. 26: 561-571; Igarashi et al., 1998, Plant Mol. Biol. 36: 917-927).

A variety of promoters specifically active in vegetative tissues, such as leaves, stems, roots and tubers, can also be used to express the nucleic acids used in the methods of the invention. For example, promoters controlling patatin, the major storage protein of the potato tuber, can be used (Martin et al., 1997, Plant J. 11: 53-62). The ORF13 promoter from Agrobacterium rhizogenes which exhibits high activity in roots can also be used (Hansen et al., 1997, Mol. Gen. Genet. 254: 337-343). Other useful vegetative tissue-specific promoters include: the tarin promoter of the gene encoding a globulin from a major taro (Colocasia esculenta L. Schott) corm protein family, tarin (Bezerra et al., 1995, Plant Mol. Biol. 28: 137-144); the curculin promoter active during taro corm development (de Castro et al., 1992, Plant Cell 4: 1549-1559) and the promoter for the tobacco root specific gene TobRB7, whose expression is localized to root meristem and immature central cylinder regions (Yamamoto et al., 1991, Plant Cell 3: 371-382).

Leaf-specific promoters, such as the ribulose biphosphate carboxylase/oxygenase small subunit (“Rubisco”) promoter, can be used. For example, the tomato RBCS1, RBCS2 and RBCS3A genes are expressed in leaves and light grown seedlings, only RBCS1 and RBCS2 are expressed in developing tomato fruits (Meier et al., 1997, FEBS Lett. 415: 91-95). Another leaf-specific promoter is the light harvesting chlorophyll a/b binding protein gene promoter (Casal et al., 1998, Plant Physiol. 116: 1533-1538). The Arabidopsis myb-related gene promoter (Atmyb5) described by Li et al., 1996, FEBS Lett. 379: 117-121, is leaf-specific. A leaf promoter identified in maize by Busk et al., 1997, Plant J. 11: 1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, can be used (Di Laurenzio et al., 1996, Cell 86: 423-433; Long et al., 1996, Nature 379: 66-69). Another useful promoter is that which controls the expression of 3-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (Enjuto et al., 1995, Plant Cell 7: 517-527). Also useful are kn1-related genes from maize and other species which show meristem-specific expression (Granger et al., 1996, Plant Mol. Biol. 31: 373-378; Kerstetter et al., 1994, Plant Cell 6: 1877-1887). Similarly, the KNAT1 promoter from Arabidopsis, whose transcript is localized primarily to the shoot apical meristem and to the inflorescence stem cortex, can be used (Lincoln et al., 1994, Plant Cell 6: 1859-1876).

One of ordinary skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

In another embodiment, a nucleic acid described in the present invention is expressed through a transposable element. This allows for constitutive, yet periodic and infrequent expression of the constitutively active polypeptide. The invention also provides for use of tissue-specific promoters derived from viruses which can include, e.g., the tobamovirus subgenomic promoter (Kumagai et al., 1995, Proc. Natl. Acad. Sci. USA 92: 1679-1683), the rice tungro bacilliform virus (RTBV), which replicates only in phloem cells in infected rice plants, with its promoter which drives strong phloem specific reporter gene expression; the cassaya vein mosaic virus (CVMV) promoter, with highest activity in vascular elements, in leaf mesophyll cells, and in root tips (Verdaguer et al., 1996, Plant Mol. Biol. 31: 1129-1139).

The present invention provides native promoters from Arabidopsis thaliana. In particular, the present invention provides drought resistance promoters from Arabidopsis capable of controlling expression of the genes of the present invention. A drought resistance promoter from Arabidopsis is a promoter derived from an Arabidopsis drought resistance gene, such as ATHK1, e.g., by cloning, isolating or modifying a native promoter from a drought resistance gene. The provided promoters can be used to initiate gene expression in a plant cell.

Preferred promoters of the present invention can control expression of the ATHK1 gene. Accordingly, the preferred promoters can control expression of genes comprising coding regions that have substantial identity to the coding region of ATHK1, e.g., preferably at least 70%, at least 80%, at least 90%, or at least 95%, 95%, 97%, 98%, 99%, or 100% identity to the coding regions of ATHK1.

A promoter sequence of the present invention can be identified, for example, by analyzing the 5′, or in some instances 3′, region of a genomic clone corresponding to the ATHK1 gene described here (GenBank Accession Number AB010914). A promoter sequence of the present invention can also be identified by analyzing the 5′ region, or in some instances 3′ region, of a gene of the present invention. Sequence characteristic of promoter sequences can be used to identify the promoter. Sequences controlling eukaryotic gene expression have been extensively studied. For instance, promoter sequence elements include the TATA box consensus sequence (TATAAT), which is upstream of the transcription start site. In most instances the TATA box is required for accurate transcription initiation. In plants, further upstream from the TATA box, there is typically a promoter element with a series of adenines surrounding the trinucleotide G (or T) N G (Messing et al., 1983, In: Genetic Engineering of Plants, pp. 221-227, Kosage, Meredith and Hollaender, eds., Springer). A number of methods are known to those of skill in the art for identifying and characterizing promoter regions in plant genomic DNA (Jordano et al., 1989, Plant Cell 1: 855-866; Meier et al., 1991, Plant Cell 3: 309-316; Zhang et al., 1996, Plant Physiol. 110: 1069-1079).

The present invention provides expression cassettes for plants or vectors, host cells, or transgenic plants comprising expression cassettes for plants or vectors comprising a promoter operably linked to a nucleic acid of the present invention. The promoters and nucleic acids can be operably linked using standard recombinant techniques. The promoter may be homologous or heterologous to the nucleic acid. Preferably, expression of the nucleic acids of the present invention under the control of the promoter will increase survival of the plant in response to drought. Promoter activity can be measured, for example, by growing the plants on media containing an osmotic substance, e.g. sorbitol, and measuring the difference in mRNA levels transcribed by genes under the control of the promoter.

Methods of Producing Drought-Resistant Plants

The present invention provides methods for enhancing drought resistance in a plant. In one embodiment of the invention, drought resistance is enhanced by increasing expression of a gene of the present invention in a plant. Thus, the methods may include introducing a construct including a promoter operably linked to a polynucleotide at least 80% identical to the sequence of ATHK1, and optionally, selecting for a plant with a phenotype associated with enhanced drought resistance. A plant with enhanced drought resistance will be healthier and live longer than a wild-type plant when exposed to drought or to a drought-causing agent. Enhanced drought resistance can be measured according to any method known to those of skill in the art. For example, a drought symptom in a test plant can be compared to a drought symptom in a control plant following exposure to drought stress, e.g. contact with a non-metabolizable sugar such as sorbitol, mannitol, polyethylene glycol (PEG), etc.

A transgenic plant having enhanced or increased expression of a gene identical or substantially identical to a preferred polynucleotide of the present invention, e.g., ATHK1, will typically display a phenotype associated with increased drought resistance to a drought-causing agent, e.g., lack of available water, increased concentration of salt and other osmotic substances, etc. Phenotypes associated with enhanced drought resistance to drought-causing agents can include, for example, plants with extended photosynthetic life cycles, plants with leaves that stay green for a longer duration of time, plants with an increased yield of fruit or vegetative part (e.g. tuber), plants with larger fruit, flowers, leaves, or stems, plants with improved storageability of the tuber or other agriculturally or horticulturally significant part, and/or plants substantially lacking in drought stress symptoms, e.g. wilting, decrease in growth or no growth, as compared to a wild type plant following exposure to drought stress.

Using specific promoters, the skilled practitioner can direct the expression of a gene of the present invention and create plants with enhanced resistance to drought. For example, in some embodiments of the present invention, a tissue-specific promoter can be used to create a transgenic plant with increased resistance to drought. Similarly, the skilled practitioner can choose from a variety of known promoters, whether constitutive, inducible, tissue-specific, and the like to drive expression of a polynucleotide of the present invention, thereby enhancing drought resistance in a plant. The sequences described herein can be used to prepare expression cassettes that enhance or increase endogenous or exogenous gene expression.

Any phenotypic characteristic reflective of enhanced drought resistance in a plant can be selected for in the present invention. For example, after introducing a polynucleotide of the present invention operably linked to a desirable promoter in a plant and regenerating the plant by standard procedures, a skilled practitioner can use standard methods to determine if the transgenic plant is a transgenic plant of the present invention, e.g., by comparing the transgenic plant to a wild type plant after exposure to drought conditions and looking for phenotypes associated with an alteration of drought resistance, e.g., wilting, chlorosis, smaller leaves, premature leaf desiccation, flowers failing to open properly, fruit dropping prematurely, fruit and seed production may be reduced or absent, etc.

A preferred phenotypic test for determining increased or enhanced drought resistance includes comparing the germination and growth of transgenic seedlings overexpressing ATHK1 to the germination and growth of untransformed control seedlings, as indicated in the examples section below. Briefly, seedlings are sown on MS plates with or without drought stress-causing agent (i.e., osmoticum such as mannitol, sorbitol, polyethylene glycol, etc.), stratified at 4° C. for 3 days, transferred to light at 22° C., and scored after 5 days growth in light at 22° C. Any phenotypic characteristic such as germination rate, root growth, shoot growth, and/or controlled deterioration test (CDT) assays can be then used to phenotypically characterize the presence (if any) of increased or enhanced drought resistance or tolerance.

Any number of means well known in the art can be used to increase activity of a gene of the present invention in a plant. Any organ can be targeted for overexpression of a protein of the present invention such as shoot vegetative organs/structures (e.g., leaves, stems, and tubers), roots, flowers, and floral or reproductive organs/structures (e.g., bracts, sepals, petals, stamens, carpels, anthers and ovules), seed (including embryo, endosperm, and seed coat), and fruit. Vascular or provascular tissues may be targeted. Alternatively, one or several genes described in the present invention may be expressed constitutively (e.g., using the CaMV 35S promoter).

Using standard methods, the skilled practitioner can perform a functional assay to determine if expression or synthesis of the putative genes or proteins confers drought resistance in a plant. Screening a transgenic plant containing a putative drought resistance gene can be performed in a variety of ways. In one example, after transformation of a plant cell with a putative polynucleotide of the present invention and subsequent cultivation of the cell, the resultant transgenic plant and a control plant are subjected to drought stress, e.g. by growing them on media containing an osmotic substance such as mannitol or sorbitol, and comparing the phenotypic characteristics, to identify the phenotype associated with enhanced drought resistance.

In one example, after introduction of the expression cassette into plants, the plants are screened for the presence of the transgene and crossed to an inbred or hybrid line. Progeny plants are then screened for the presence of the transgene and self-pollinated. Progeny from the self-pollinated plants are grown. The resultant transgenic plants can be examined for any of the phenotypic characteristics associated with enhanced drought resistance characteristics, e.g., healthier leaves following exposure to an osmotic substance. Using known procedures, one of skill can screen for plants of the invention by detecting increased or decreased levels of ATHK1 transcript and/or protein in a plant and detecting the desired phenotype. Means for detecting and quantifying mRNA or proteins are well known in the art, e.g., Northern Blots, QRT-PCR, DNA microarrays, Western Blots or protein activity assays.

Gene amplification and/or expression can be measured in a sample directly, for example, by conventional Southern blotting, Northern blotting to quantitate the transcription of mRNA, dot blotting (DNA analysis), DNA microarrays, or in situ hybridization, using an appropriately labeled probe, based on the sequences provided herein. Various labels can be employed, most commonly radioisotopes, particularly ³²P. However, other techniques can also be employed, such as using biotin-modified nucleotides for introduction into a polynucleotide. The biotin then serves as the site for binding to avidin or antibodies, which can be labeled with a wide variety of labels, such as radionuclides, fluorescers, enzymes, or the like. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. Gene expression can also be measured using DNA microarrays, commonly known as gene chips.

The polypeptides of the present invention may be used alone or in combination with other proteins or agents to enhance drought resistance. Examples of such agents include, for example, the Lily ASR Protein that confers drought and salt resistance in Arabidopsis (Yang et al., 2005, Plant Physiol. 139: 836-846) and other proteins that help plants tolerate arid conditions and can be used to produce hardier crop varieties (Moffat et al., 2002, Science 296: 1226-1229); application of certain nutrients, fertilizers, etc.

Transgenic Plants with Enhanced Drought Resistance

The present invention also provides transgenic plants that have enhanced drought resistance. In one aspect of the present invention, transgenic plants of the present invention include recombinant expression cassettes comprising a promoter operably linked to a nucleic acid of the present invention. The nucleic acid can be operably linked to the promoter sequence in a sense or antisense orientation.

Enhancing or increasing expression of a gene of the present invention in a plant may modulate drought resistant processes by a variety of pathways. The particular pathway used to modulate drought resistance is not critical to the present invention.

DNA constructs of the invention may be introduced into the genome of a desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Agrobacterium tumefaciens-mediated transformation techniques, including disarming and use of binary vectors, are well described in the scientific literature (Horsch et al., 1984, Science 233: 496-498; Hellens et al., 2000, Trends Plant Sci. 5: 446-451; Gelvin, 2003, Microb. and Mol. Biol. Reviews 67: 16-37). Other species of bacteria outside the Agrobacterium genus can also be used for gene transfer into plants (Broothaerts et al., 2005, Nature 433: 629-633).

Microinjection techniques are known in the art and well described in the literature. The introduction of DNA constructs using polyethylene glycol precipitation is described in Paszkowski et al., 1984, EMBO J. 3: 2717-2722. Electroporation techniques are described in Fromm et al., 1985, Proc. Natl. Acad. Sci. USA 82: 5824-5828. Biolistic transformation techniques are described in Klein et al., 1987, Nature 327: 70-73.

Transformed plant cells that are derived by any of the above transformation techniques can be cultured to regenerate a whole plant which possesses the transformed genotype and thus the desired phenotype. Regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Binding, 1985, Regeneration of Plants, Plant Protoplasts, pp. 21-73, CRC Press, Boca Raton, Fla. Regeneration can also be obtained from plant callus, explants, organs, or parts thereof, using regeneration techniques described generally in Klee et al., 1987, Annu. Rev. Plant Physiol. 38: 467-486.

The nucleic acids of the invention can be used to confer expected desired traits, i.e., drought resistance, on essentially any plant. Thus, the invention has use over a broad range of plants, monocots and dicots, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and Zea. Examples include tobacco and Arabidopsis, cereal crops such as maize, wheat, rice, soybean barley, rye, oats, sorghum, alfalfa, clover and the like, oil-producing plants such as canola, safflower, sunflower, peanut and the like, vegetable crops such as tomato tomatillo, potato, pepper, eggplant, sugar beet, carrot, cucumber, lettuce, pea and the like, horticultural plants such as aster, begonia, chrysanthemum, delphinium, zinnia, lawn and turfgrasses and the like. In another embodiment, a homolog of ATHK1 gene may be used to create a nucleic acid construct for overexpression in a broad range of plants, in a manner described herein.

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

### EXAMPLES

### T-DNA Mutant Screen and Identification

Using a PCR-based strategy, T-DNA mutagenized populations of Arabidopsis thaliana were screened for the presence of insertions in ATHK1. The sequences of primers specific for ATHK1 were: 5′-AGGAAGGTGTTCGATAAAATGACTGAATG (SEQ ID NO:1), and 5′-CACATCCAGTATCATCAACCTCAAACCA (SEQ ID NO:2). The sequences of primers specific for the T-DNA border were: 5′-CATTTTATAATAACGCTGCGGACATCTAC (SEQ ID NO:3), and 5′-TTTCTCCATATTGACCATCATACTCATTG (SEQ ID NO:4).

DNA sequencing of PCR products confirmed the locations of the junctions of genomic and T-DNA sequences. Two independent homozygous knockout mutations were established by self-crossing and were named athk1-3 and athk1-4.

Molecular Complementation

Two fragments (one 6-kb fragment and one 8-kb fragment) containing the entire coding sequence and putative regulatory sequence of ATHK1 were amplified using high-fidelity PCR.

To amplify the 6-kb fragment, the following primers were used: 5′-CCGCTCGAGCTCTCCATTGGCCATTTTACCTTCTAC (SEQ ID NO:5), and 5′-ATAAGAATGCGGCCGCTTACCCCCAAAAACCTCATCGTCAA (SEQ ID NO:6). To amplify the 8-kb fragment, the following primers were used: 5′-CCGCTCGAGTGTTAAATCGCAGTCTATACAGTCATC (SEQ ID NO:7), and 5′-ATAAGAATGCGGCCGCGGGCTTAAAAATTGTTCCAGAGTTCG (SEQ ID NO:8).

To confirm the presence of full-length, wild-type sequence, DNA sequencing was carried out on the entire length of all clones used for rescue. Both fragments were ligated into the NotI and XhoI sites of the binary vector pGreenII0029 (Hellens et al., 2000, Trends Plant Sci. 5: 446-451). Each construct was used to transform six different athk1-3 and athk1-4 plants using an Agrobacterium tumefaciens-mediated floral dip procedure. Transformants were selected with 50 μg/mL kanamycin (Sigma, St. Louis, Mo.). Rescued plants were identified as those homozygous for an athk1 T-DNA insertion and homozygous for an ATHK1 transgene, based on PCR genotyping. Multiple rescued lines were identified for both the 6-kb and 8-kb fragments; data for a representative line is shown in all figures.

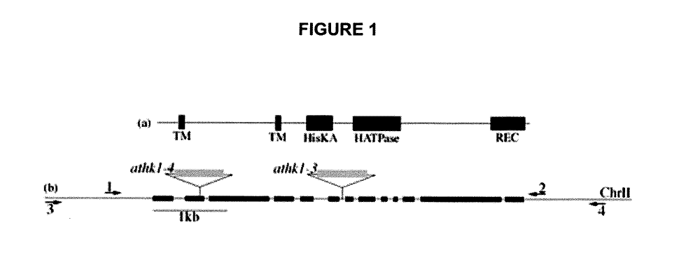

The schematic diagram of the ATHK1 protein is shown in FIG. 1(a). Numerals indicate the amino acid position of each domain. A few salient domains are shown: TMD, transmembrane domain; HisKA, histidine kinase domain; HATPase, ATP binding domain; REC, receiver domain.

Shown in FIG. 1(b) are structures of athk1 alleles and the ATHK1 fragment used for molecular complementation. The T-DNA in FIG. 1 is not drawn to scale. Two alleles of athk1 were isolated using a PCR-based strategy. Two separate primer pairs were used to amplify a genomic ATHK1 fragment used for molecular complementation. FIG. 1 illustrates how the athk1-3 allele (a3) carries a T-DNA insertion in the sixth intron, 2465 bp downstream of the ATHK1 start. The athk1-4 allele (a4) carries a T-DNA insertion in the second exon, 593 bp downstream of the ATHK1 start. For PCR of ATHK1 genomic DNA for molecular complementation, primer pair 1 (SEQ ID NO:5) and 2 (SEQ ID NO:6) was used to amplify the 6-kb fragment; primer pair 3 (SEQ ID NO:7) and 4 (SEQ ID NO:8) was used to amplify the 8-kb fragment.

Overexpression

A 3.3-kb cDNA fragment containing the entire open reading frame of ATHK1 was overexpressed under the control of the enhanced CaMV 35S promoter. The tobacco mosaic virus Ω sequence was inserted upstream of the ATHK1 sequence to increase the translational level. Wassilewskija (Ws) and athk1 plants were transformed as described above for molecular complementation. Transformants were selected with 30 μg/mL hygromycin (Sigma). Multiple overexpressing lines were identified; data for a representative line is shown in all figures.

Plant Material and Growth Conditions

Arabidopsis thaliana (L.) ecotype Wassilewskija (Ws) was used for all experiments. Seeds were sown on media (pH 5.7) containing one-half-strength Murashige and Skoog (MS) salts (Sigma), 2.5 mM MES, 1% (w/v) sucrose, and 0.8% (w/v) washed agar. Plates were stratified at 4° C. for 3 days, and then transferred to light (42 μmol m⁻² s⁻¹). Seedlings were transferred to soil after 10 days growth on MS plates. Plants were housed under the following growth conditions: 23° C., 24 hour light, and ˜60% humidity. After harvesting and approximately 3 weeks after-ripening, seeds were stored at 4° C.

Sorbitol, mannitol, glucose, and sucrose plates were made with one-half-strength MS media by adding autoclaved sugar solutions after the medium had cooled to approximately 55° C. NaCl plates were made with one-half-strength MS media by adding solid NaCl directly to media before autoclaving. For ABA assays, ethanolic stock solutions of ABA (Sigma) were made at 1000× strength and added to one-half-strength MS media after the medium had cooled to approximately 55° C. Ethanolic stock solutions of fluridone (Chem Service, West Chester, Pa.) were freshly prepared for each experiment.

Water and ABA Stress Assays

For all assays, seeds of simultaneously produced and harvested lots were compared. For drought assays, sterilized seeds were sown on MS plates and were cold-treated in the dark at 4° C. for 3 days. After 7 days growth in constant light, seedlings were transferred to soil and grown for an additional 7 days in short day conditions (8 hour light/16 hour dark), with water every 3 days. Prior to drought treatment, plants were saturated with water and then transferred to dry conditions. Plants were withheld from water and observed daily for signs of wilting. Plants were photographed after approximately 6 weeks when the largest differences between genotypes were apparent.

For osmotic stress germination assays, sterilized seeds were sown on MS plates containing sorbitol, mannitol, sucrose, or glucose and were cold-treated in the dark at 4° C. for 3 days. Plates were exposed to light for 1 h at 23° C. and scored after 5 days growth. At least three replications of 100 seeds per line were tested for all treatments. For ABA germination assays, sterilized seeds were sown on MS plates containing ABA and were cold-treated in the dark at 4° C. for 3 days. Plates were exposed to light for 1 hour at 23° C. and scored after 5 days growth in darkness. Germination was indicated by clear protrusion of the radicle. For root growth assays, sterilized seeds were sown on MS plates and were cold treated at 4° C. for 3 days. Plates were grown vertically in constant light for 3 days at 23° C. and then transferred to MS plates supplemented with different concentrations of sorbitol, and scored after 7 days additional growth. At least three replications of 20 roots per line were tested for each treatment.

Stomatal Assays

For stomatal assays, sterilized seeds were sown on MS plates and were cold-treated in the dark at 4° C. for 3 days. After 7 days growth in constant light, seedlings were transferred to soil and grown for an additional 5 weeks in short day conditions (8 hour light/16 hour dark), with water every 3 to 5 days. For inhibition of stomatal opening, adult rosette leaves were harvested immediately prior to onset of light, and placed in the dark at room temperature for 2 hours in a solution of 10 mM KCl, 7.5 mM iminodiacetatic acid, and 10 mM MES (pH 6.15). ABA was added to the solution to a final concentration of 30 μM from 1000× ethanolic stock solutions, and an equivalent amount of ethanol was added to the controls. Leaves were placed in bright light at room temperature for 2 hours. For baseline measurements, stomata were measured after dark treatment. To measure stomata, epidermal peels were taken from leaves and imaged at 400× magnification under bright field microscopy. Images were captured by a SPOT Insight CCD camera, and stomatal apertures were measured in NIH Image.

Controlled Deterioration (CD) Test

A CD test simulates maturation of seeds under controlled conditions and can thus reveal the relative storage potential of seeds (Tesnier et al., 2002, Seed Sci. and Technol. 30: 149-165). Seeds were equilibrated at 85% relative humidity and stored for 4 days at 60° C., for artificial ageing of seeds. Seeds were then allowed to equilibrate at −30% humidity. For germination assays, seeds were sown on filter paper moistened with water, stratified for 3 days at 4° C., transferred to light, and scored after 5 days growth at 23° C. At least three replications of 100 seeds per line were tested.

qRT-PCR

For RNA analysis on seedlings, plants were grown for three days on chromatography paper overlaid on media (pH 5.7) containing one-half-strength MS, 2.5 mM MES, and 0.8% (w/v) agar. For stress treatment, seedlings were transferred along with the chromatography paper to liquid one-half-strength MS, 2.5 mM MES ±300 mM sorbitol for 16 hours, after which time tissue was collected. Whole seedlings were ground to a fine powder using a Mixer Mill (Retsch) in RNase-free conditions. Total RNA was prepared using the RNeasy Plant Mini Kit (Qiagen) according to the manufacturer's instructions. For RNA analysis on seeds, dry seeds were frozen in liquid N₂ and ground to a fine powder using a mortar and pestle in RNase-free conditions. Seed RNA was extracted as described by Vicient and Delseny, 1999, Anal. Biochem. 268: 412-413). All RNA samples were DNase treated (Promega) prior to cDNA synthesis. 3 μg of DNase-treated RNA was used for first-strand cDNA synthesis with Superscript II (Invitrogen) using an oligo dT (dT₂₄+V) primer, with the following modifications to the manufacturer's instructions: the oligo dT primer (100 μM) was mixed with RNA in 12 μL of RNase-free water and was heated to 70° C. for 10 min prior to addition of first-strand buffer and DTT.

Following first-strand synthesis, optimal cDNA template amount was determined by preparing a dilution series. For final analyses, 2 μL of a 250-fold dilution of the RT reaction (corresponding to 2.4 ng RNA) was used for qPCR amplification with SYBR Premix Ex Taq (Takara), with the following modifications to the manufacturer's instructions: amplifications were performed in 20 μL reaction volumes with 5 μmol of each primer. qPCR reactions were performed with the iCycler Real Time PCR System (BioRad). The genes selected for qRT-PCR were AtEm1 (At3g51810), AtEm6 (At2g40170), RAB18 (At5g66400), ABI5 (At2g36270), FUS3, (At3g26790), LEC1 (At1g21970), ABA1 (At5g67030), ABA2 (At1g52340), AAO3 (At2g27150), NCED3 (At3g14440), ZAT10 (At1g27730), RD29B (At5g52300), LEA14 (At1g01470), KIN1 (At5g15960), and ATHK1 (At2g17820). Primers are listed in the 5′ to 3′ direction. Primers were designed around introns to have a cDNA product approximately 100 to 300 bp long. Primer melting temperatures were calculated using the program PrimerSelect in the DNAStar Lasergene software package. Optimal annealing temperatures for each primer pair were determined experimentally.

Table 1 lists the sequences of primers used for qRT-PCR analysis. The forward (F) and reverse (R) sequences of the primers used for amplifications were: ATHK1 (At2g17820): F, 5′-CTAGTTTACGGAATTCCTCGCCTTATGAC-3′ (SEQ ID NO:9); R, 5′-CTCTGTGCCAATCTCTGCTCTCCTTATCG-3′ (SEQ ID NO:10); CRA1 (At5g44120): F, 5′-AGCGCCAGGTGCACCGATAAC-3′ (SEQ ID NO:11); R, 5′-GTTCGCGTTTGCGTTCCACTG-3′ (SEQ ID NO:12); CRU2 (At1g03880): F, 5′-TCTCCGCCTTAGCGCTCTTCGTG-3′ (SEQ ID NO:13); R, 5′-CCTGTGCGTTTTCGTTTGTCTTGA-3′ (SEQ ID NO:14); CRU3 (At4g28520): F, 5′-CGACATCGCCAACTACCAAAACCA-3′ (SEQ ID NO:15); R, 5′-CGTCGAACCCGCTCCACAAGT-3′ (SEQ ID NO:16); 2SSSP1 (At4g27140): F, 5′-ATCTACCGCACCGTCGTTGAGTTC-3′ (SEQ ID NO:17); R, 5′-GCCTTGCCTTGCTTGCTGGAG-3′ (SEQ ID NO:18); 2SSSP2 (At4g27150): F, 5′-TGGCAAACAAGCTCTTCCTCGTCT-3′ (SEQ ID NO:19); R, 5′-GGGTTGCTGGCGTCATCTTCGTC-3′ (SEQ ID NO:20); 2SSSP4 (At4g27170): F, 5′-CGCAAGCAAATGTGGCAAGGAC-3′ (SEQ ID NO:21); R, 5′-AGGTGGGGCAAACGCAAACTG-3′ (SEQ ID NO:22); ACT2 (At3g18780): F, 5′-GCATGAAGATCAAGGTGGTTGCAC-3′ (SEQ ID NO:23); R, 5′-ATGGACCTGACTCATCGTACTCACT-3′ (SEQ ID NO:24).

Presence of a single PCR product was verified by melt-curve analysis. Relative quantification of gene expression was carried out using the Real-Time PCR Miner algorithm (Zhao et al., 2005, J. Comput. Biol. 12: 1047-1064) to account for differences in PCR efficiencies of different primer pairs used for different genes. All reactions were performed in quadruplicate on separate plates. Expression levels were normalized using values obtained for the housekeeping gene ACT2 (At3g18780). Values were typically compared to wild-type under control conditions.

TABLE 1
Sequences of primers used for qRT-PCR analysis

| | | | | | | | |
|---|---|---|---|---|---|---|---|
| | | | | | | cDNA | gDNA |
| Gene | Name | Sequence | Tₘ(° C.) | % GC | Length | Product | Product |

| | | | | | | | |
|---|---|---|---|---|---|---|---|
| AtEm1 | AtEm1-RTF1 | ATGGCGTCAAAGCAACTGAGCAGA | 61.8 | 50.0 | 24 | 414 | 598 |
| | AtEm1-RTR2 | TCCGCACGCTCTCCACCAGATTT | 63.6 | 56.5 | 23 | | |
| AtEm6 | AtEm6-RTF1 | AAAGAAGGGCGAGACCGTCGTG | 61.2 | 59.1 | 22 | 185 | 282 |
| | AtEm6-RTR1 | GTGTTCCCCACCAGGCTTGTCTC | 60.6 | 60.9 | 23 | | |
| RAB18 | RAB18-RTF1 | AGGTGGCCAAGGATACGGAACAGG | 63.0 | 58.3 | 24 | 321 | 402 |
| | RAB18-RTR1 | GCCCATCGCTTGAGCTTGACCAG | 63.7 | 60.9 | 23 | | |
| ABI5 | ABI5-RTF2 | ATCCAAACCCGAACCAAAACCAA | 60.1 | 43.5 | 23 | 543 | 711 |
| | ABI5-RTR2 | CTTCCTCTCCAACTCCGCCAATG | 60.7 | 56.5 | 23 | | |
| FUS3 | FUS3-RTF1 | GGGTTATCGGCGTCTGTGCCTCTT | 63.2 | 58.3 | 24 | 252 | 524 |
| | FUS3-RTR1 | TCCGCGGCTTTCTTCGGGAGTA | 63.6 | 59.1 | 22 | | |
| LEC1 | LEC1-RTF1 | CGGCGCCGGTGACAAGAAC | 60.9 | 68.4 | 19 | 555 | 555 |
| | LEC1-RTR1 | GACGAAGAGCCACCACCAACACTG | 61.6 | 58.3 | 24 | | |
| ABA1 | ABA1-RTF1 | GTGGGGCTGATGCTCCAAATGG | 62.1 | 59.1 | 22 | 180 | 265 |
| | ABA1-RTR1 | AATCCCCGAGCAGCGTAACACG | 61.9 | 59.1 | 22 | | |
| ABA2 | ABA2-RTF1 | ATGTGGAGCACCGTGCCCTGATA | 62.2 | 56.5 | 23 | 182 | 182 |
| | ABA2-RTR1 | GACCAACGCCTCCCACAACACC | 62.1 | 63.6 | 22 | | |
| AAO3 | AAO3-RTF1 | GAAGAGTTGGTGGTGGCTTTGGTG | 60.0 | 54.2 | 24 | 202 | 287 |
| | AAO3-RTR1 | TCCAGTGCTGTGAGCTTCCCATCT | 60.9 | 54.2 | 24 | | |
| NCED3 | NCED3-RTF1 | CCCGTCCGGCGTAATCTTCC | 60.1 | 65.0 | 20 | 76 | 76 |
| | NCED3-RTR1 | TAGCTCCGTTGCGCACATACACTC | 60.2 | 54.2 | 24 | | |
| ZAT10 | STZ-RTF1 | TGGCGCTCGAGGCTCTTACATC | 60.3 | 59.1 | 22 | 219 | 219 |
| | STZ-RTR1 | CCGGAGGAGGAGGAGGCTGACG | 64.9 | 72.7 | 22 | | |
| RD29B | RD29B-RTF1 | GAGGAGGATCGGATTATCTCAGTGGTGTAT | 61.8 | 46.7 | 30 | 171 | 241 |
| | RD298-RTR1 | AGTCTTCTTCGCGTCCTTGTCTTGATTTCT | 63.6 | 43.3 | 30 | | |
| LEA14 | LEA14-RTF1 | GGCCAAAGTCTCTGTCACCAATCCT | 60.5 | 52.0 | 25 | 141 | 217 |
| | LEA14-RTR1 | GCCGTCATGTCCTTAGCTTTCAGAGA | 60.9 | 50.0 | 26 | | |
| KIN1 | KIN1-RTF1 | ACAAGAATGCCTTCCAAGCCGGTCAGAC | 67.5 | 53.6 | 28 | 131 | 412 |
| | KIN1-RTR1 | CGATACACTCTTTCCCGCCTGTTGTGCT | 66.6 | 53.6 | 28 | | |
| ATHK1 | RTF7 | CTAGTTTACGGAATTCCTCGCCTTATGAC | 60.7 | 44.8 | 29 | 113 | 184 |
| | RTR7 | CTCTGTGCCAATCTCTGCTCTCCTTATCG | 64.2 | 51.7 | 29 | | |

ABA Measurements

For ABA measurements on sorbitol treated seedlings, plants were prepared as for qRT-PCR measurements. For ABA measurements on seeds, seeds were sown on two layers of chromatography paper moistened with sterile water. Seeds were cold-treated in the dark for 3 days at 4° C. Seeds were transferred to light and collected after 16 h, 24 h, and 48 h. Dry seeds were used as the control. The extraction procedure was performed as described (Chiwocha et al., 2003, Plant J. 35: 405-417), with the following modifications: following extraction in 99% isopropanol and 1% acetic acid, the extract was dried in a SpeedVac. The pellet was resuspended in 15% methanol and 1% acetic acid and then passed through a Sep-Pak C18 column as described. The purified extract was dried in a SpeedVac and reconstituted with 30 μL of 15 μM ammonium acetate. For each sample, 10 μL was used for RP-HPLC/ESI-MS/MS and the eluting ions were measured with MRM. The level of ABA in the samples was quantified in relation to their internal standard using calibration curves that had been generated for each compound. Each experiment was carried out twice, with three biological replicates.

Transcriptional Profiling

To generate samples to be used for microarray analysis, plants were grown on circles of filter paper on media containing half-strength MS, 2.5 mM MES, and 0.8% (w/v) agar. Three-day old seedlings were transferred to a liquid solution of half-strength MS, 2.5 mM MES, with or without 300 mM sorbitol. After 16 h of sorbitol treatment, tissue was harvested by pooling plates for each genotype and treatment, and freezing in liquid N₂. Tissue was homogenized under liquid N₂. The Qiagen Plant RNeasy kit was used for total RNA extraction. Data from five biological replicates was averaged and is presented here. For microarray analysis, fragmented, biotin-labeled cRNA was synthesized according to Affymetrix protocols. The GeneChip Eukaryotic Poly-A RNA control kit was used to provide positive controls to monitor the entire labeling process.

Affymetrix ATH1 whole genome Arabidopsis thaliana microarrays were used to generate expression data. Eight samples were tested in this experiment: (1) wild-type, (2) wild-type+300 mM sorbitol, (3) athk1-3, (4) athk1-3+300 mM sorbitol, (5) athk1+ATHK1, (6) athk1+ATHK1+300 mM sorbitol, (7) 35S ATHK1, and (8) 35S ATHK1+300 mM sorbitol. Three biological replicates of each sample were prepared, for a total of 24 microarrays. Probe-level expression data was normalized using the robust multi-array average (RMA) method.

ATHK1 Functions in Osmotic Stress Tolerance

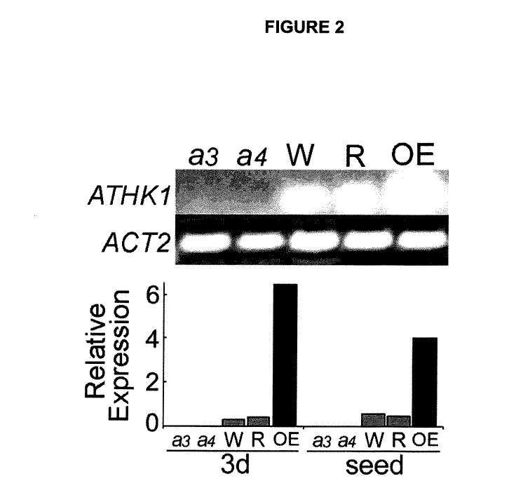

ATHK1 mutants display altered water stress sensitivities. To analyze the role of ATHK1 in plants, four genotypes of plants with altered ATHK1 expression levels were used: (1) wild-type (Ws), (2) two different athk1 null mutants, athk1-3 and athk1-4, (3) ATHK1-rescued athk1 mutants, and (4) 35S ATHK1 overexpressors. athk1-3 contains a T-DNA insertion within the sixth intron, 2465 bases downstream of the translation start site, and athk1-4 contains a T-DNA insertion within the second exon, 593 bases downstream of the translation start site (FIG. 1). Expression of ATHK1 in both athk1 null mutants was undetectable by standard RT-PCR analysis or by QRT-PCR (FIG. 2), so it was concluded that both alleles represent nulls. Four independently derived lines of ATHK1-rescued athk1-3 or athk1-4 mutants were examined.

FIG. 2 shows relative expression levels of ATHK1 in seeds and three-day-old seedlings. The legends indicate: wild-type (W; dark grey), athk1-3 (a3; light grey), athk1-4 (a4; light grey), athk1/ATHK1 rescued (R; dark grey), and 35S:ATHK1 overexpressor (OE; black). RNA extracted from three-day old seedlings was used for standard RT-PCR and quantitative RT-PCR. RNA extracted from mature seeds was used for quantitative RT-PCR. All quantifications were made in quadruplicate. The expression levels shown are relative to those of Ws plants for each developmental stage. Expression levels were also normalized to the housekeeping gene ACT2. Error bars indicate standard deviations.

Levels of ATHK1 RNA in the rescued lines were comparable to that of wild-type. Five independently derived lines of 35S:ATHK1 overexpressors were examined. Levels of ATHK1 RNA in these overexpressors were between 2- and 12-fold greater than that of wild-type. Data from an overexpressing line with 5-fold increase in ATHK1 expression (FIG. 2) is presented in all figures.

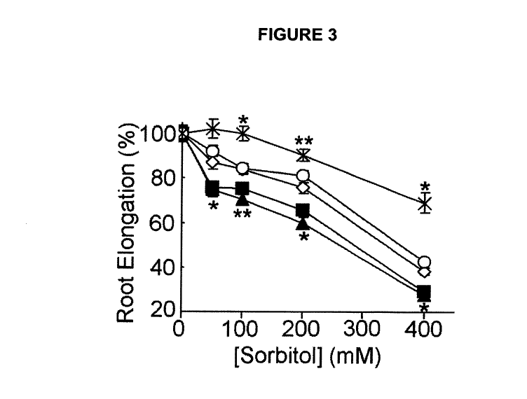

To test osmotic sensitivity during early plant growth, seed germination and root elongation were assayed at various concentrations of the non-metabolizable sugar sorbitol. FIG. 3 shows a graph illustrating the altered osmotic response of ATHK1 alleles during root elongation. Wild-type (open diamonds), athk1-3 (closed squares), athk1-4 (closed triangles), athk1/ATHK1 rescued (open circles), and a 35S:ATHK1 overexpressor (asterisks) from matched seed lots were scored for root elongation on the indicated concentrations of sorbitol after five days of stress treatment. Each value represents the mean percent elongation for at least three replicates of at least 20 roots. Error bars represent the SE. Asterisks represent significance based on a two-tailed t-test (*p<0.01, **p<0.05).

As can be seen in FIG. 3, athk1 null mutants are hypersensitive to sorbitol during root elongation. Introduction of a wild-type copy of ATHK1 rescues this osmotic sensitivity back to wild-type levels. Overexpression of ATHK1 results in plants that are more resistant than wild-type to osmotic stress in during root elongation. Similar phenotypes were obtained with sucrose, glucose, and the non-metabolizable sugar mannitol (data not shown), indicating a defect in detection or response to osmolytes, rather than a specific sugar.

ATHK1 Functions in Seed Desiccation

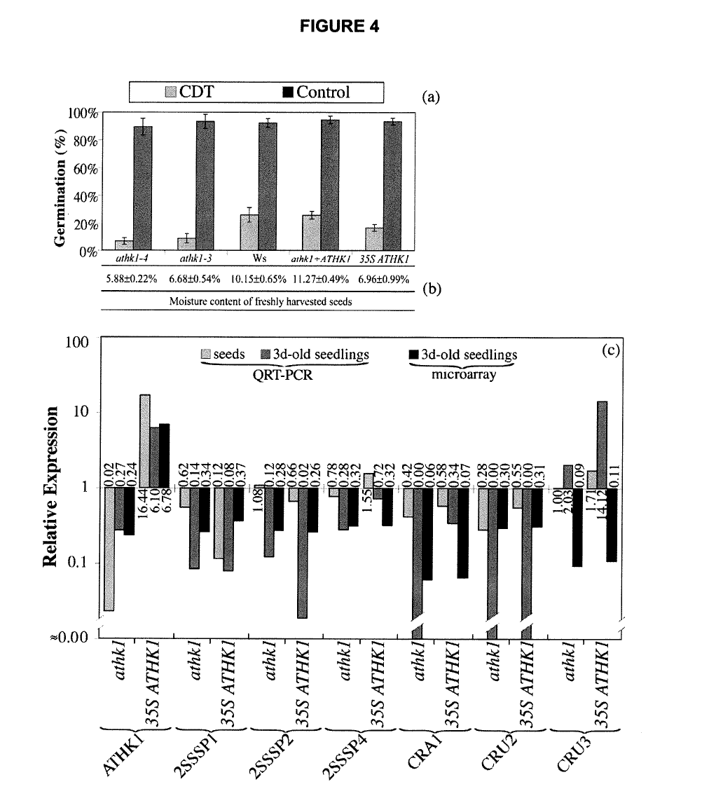

The data obtained from tests of various seed phenotypes is presented in FIG. 4. Shown in FIG. 4(a) is the viability of athk1 seeds. Seeds were either subjected to a controlled deterioration test (CDT), a test commonly used to assess seed longevity (Tesnier et al., 2002, Seed Sci. Technol. 30: 149-165) (light grey columns), or left untreated as a control (dark grey columns). Each value is the average seed percent germination for at least 3 replicates of at least 100 seeds. Error bars indicate standard errors.

Shown in FIG. 4(b) is the seed moisture content for athk1-4, athk1-3, Ws, athk1+ATHK1, and 35S ATHK1. Approximately 200 mg of seeds were dried at 65° C. for 16 h and their mass before and after drying was compared. Four independently harvested seed lots were averaged. Error indicates standard errors.

Shown in FIG. 4(c) are the seed transcript levels. Microarray or QRT-PCR analysis was carried out on seeds and three-day-old seedlings to measure the levels of important seed storage compounds. All quantifications were made in triplicate. The expression levels shown are relative to a wild-type control for each developmental stage. For QRT-PCR, expression levels were also normalized to an ACT2 control gene.

Homozygous athk1 seeds began to lose the ability to germinate after approximately six months of dry storage, conditions that do not affect wild-type seeds. This effect was exacerbated by high temperature and high humidity, conditions of a controlled deterioration test (CDT) that artificially age seeds (FIG. 4a). The loss of seed viability over time may be caused by defects in the regulation of embryo desiccation, since mutant athk1 seeds showed a significantly smaller amount of moisture than wild-type or ATHK1 rescued seeds (FIG. 4b). Overexpression of ATHK1 in seeds caused defects similar to those of the athk1 null mutants, suggesting that regulation of the levels of ATHK1 protein is very important for seed quality.

Furthermore, a number of seed specific transcripts were identified as significantly down-regulated in athk1 null mutants, suggesting possible targets of ATHK1 regulation, and a molecular explanation for the observed seed defects in athk1 plants. To further confirm the expression levels of these transcripts in seeds, QRT-PCR analysis on RNA extracted from athk1 seeds was performed (FIG. 4c). In all cases, these seed storage transcripts were confirmed to have much reduced expression in both athk1 null and 35S ATHK1 plants, suggesting that reduced levels of storage proteins might also contribute to the observed reduced viability in mutant seeds.

The Role of ATHK1 in Seed Maturation

It has generally been accepted that the rise of ABA levels in seeds during maturation correlates directly with reduction in seed water content. Thus, the reduced ABA levels and increased moisture content in seeds of athk1 null mutants agrees well with information from previous studies, and suggests that ATHK1 has a role in the process of seed desiccation. It is also during this desiccation period in seed maturation when levels of LEA proteins and other seed storage components accumulate. In a plant with seed phenotypes such as reduced viability, reduced ABA, and increased water, one might also expect reduced levels of some seed specific proteins. Interestingly, the LEA transcripts AtEm1, AtEm6, and RAB18 are increased in athk1 mutant seeds. However, this apparent contradiction might be explained by the fact that ABA and transcript levels were only examined in dry seed, not throughout the process of seed maturation. The process of maturation is extremely sensitive to small alterations of ABA in seeds. Among the ATHK1 mutants, there may be variations in the timing or levels of ABA accumulation during the period of seed maturation when embryonic levels of ABA rise. Alternatively, LEA transcript levels might not accurately reflect LEA protein levels in athk1 plants. It is possible that other mechanisms, such as post-transcriptional regulation affect the accumulation of seed proteins. The transcription factor ABI5 regulates accumulation of LEA transcripts in seeds. Similar to that observed for the LEA genes, increased levels of ABI5 transcript were measured in athk1 null mutants. Not wanting to be bound by the following explanation, it is possible that ATHK1 negatively regulates the transcription factor ABI5 in seeds, which in turn influences LEA accumulation.

Furthermore, processes such as seed moisture level and LEA gene synthesis, which are known to be regulated by ABA in the seed, are altered in ATHK1 mutants, leading to further confidence that ATHK1 has some role in sensing or regulating embryo water loss, most likely through an ABA-dependent pathway. ATHK1 may play a sensory or regulatory function during seed maturation, and the exact timing or kinetics of processes such as desiccation, ABA accumulation, or LEA synthesis are altered in athk1 mutants, leading to the differences in dry seed phenotypes and dry seed levels of certain RNAs that were observed.

The precise function of the LEA proteins AtEm1 and AtEm6 is not clear, but it has been suggested that they function as osmoprotectants during the period of seed desiccation. In abi4 and abi5 mutants with reduced Em protein accumulation, germination is less sensitive to osmotic or ionic stress than wild-type and also less sensitive to ABA. In the athk1 mutants with increased ABI5, AtEm1, and AtEm6 expression, there was increased sensitivity to osmotic or ionic stress, which would correlate well with previous results. However, the athk1 mutants display decreased sensitivity to ABA, opposite of what has been demonstrated for abi4 and abi5. Therefore, the effects of the ATHK1 mutation on Em gene accumulation and osmotic stress sensitivity might be due to ATHK1 acting as a negative regulator of ABI5, while the effects of the ATHK1 mutation on ABA biosynthesis and ABA sensitivity might be due to ATHK1 acting as a positive regulator of ABA biosynthesis during osmotic stress and seed desiccation. Clearly, there is the potential for a high level of cross-talk and complex interaction between the different regulatory genes and proteins in the pathway leading to seed maturation and germination. Furthermore, like other seed maturation factors such as ABI5, ATHK1 seems to function as both a positive and negative regulator, depending on the target pathway.

Table 2 shows the viability and moisture content in wild-type and ATHK1 mutant seeds. For seed survival tests, seeds were subjected to 80% humidity and 60° C. for 4 days as a controlled deterioration (CD) treatment. Each value represents the mean percent germination for four replicates of at least 100 seeds. Error indicated is SE. For seed moisture tests, approximately 200 mg of seeds were dried at 65° C. for 16 hours, and seed mass before and after drying was measured. Measurements from four independently harvested seed lots were averaged. Error indicated is SE. Measurements from athk1-3, athk1-4, and 35S:ATHK1 are significantly different from those of wild-type (p<0.01, based on two-tailed t-test).

TABLE 2
Viability and moisture content in wild-type and ATHK1 mutant seeds

| | Ws | athk1-3 | athk1-4 | athk1/ATHK1 | 35S:ATHK1 |
|---|---|---|---|---|---|
| Survival | 7.9 ± 1.9% | 3.6 ± 2.0% | 3.7 ± 2.8% | 8.5 ± 3.2% | 13.1 ± 2.8% |
| Moisture | 12.5 ± 2.5% | 15.4 ± 2.0% | 14.2 ± 1.5% | 10.4 ± 2.1% | 9.0 ± 2.3% |

Transcriptional Analysis

To determine genes which function downstream of ATHK1 in Arabidopsis, microarray analysis on all four classes of mutants described above was performed, with and without sorbitol stress.

Table 3 shows composite data on ATHK1-regulated genes. Individual probe-level data from Affymetrix arrays was normalized using the RMA method and tested using two-factor ANOVA to determine genes significantly affected by ATHK1 transcript level and sorbitol stress (p-value α<0.01). Genes which showed no response to sorbitol in athk1 null plants and greater response in 35S ATHK1 plants are listed here and broadly grouped into functional categories.

Genes that are known to be induced by osmotic stress include those that produce metabolites important for protecting cells such as chaperones and LEA (late embryogenesis abundant) proteins, key enzymes for osmolyte biosynthesis such as proline and sucrose, and detoxification enzymes and lipid-transfer proteins. Another important group of genes induced by osmotic stress are those responsible for regulation of signal transduction and further gene expression during the stress response, such as transcription factors.

TABLE 3
ATHK1-regulated genes

| | | | | | |
|---|---|---|---|---|---|
| | | | | | 35S |
| Locus | | | Athk1 + | Ws + | ATHK1 + |
| Identification | Annotation | p-value | sorbitol | sorbitol | sorbitol |

Abiotic Stress Response

| | | | | | |
|---|---|---|---|---|---|
| At5g52300 | RD29B low-temperature- | 0.00155 | 1.78 | 3.25 | 4.52 |
| | responsive protein | | | | |
| At3g02480 | ABA-responsive protein- | 6.1E−05 | 1.22 | 3.67 | 4.26 |
| | related | | | | |
| At2g42540 | COR 15 cold-responsive | 0.00558 | 1.06 | 2.60 | 4.06 |
| | protein | | | | |
| At5g52310 | RD29A low-temperature- | 4.7E−08 | 1.76 | 2.80 | 3.68 |
| | responsive protein 78 | | | | |
| At4g11650 | ATOSM34 osmotin-like | 9.7E−05 | 1.40 | 2.28 | 3.03 |
| | protein | | | | |
| At2g42530 | COR15b cold-responsive | 3.5E−05 | 1.07 | 2.00 | 2.68 |
| | protein | | | | |

Metabolism

| | | | | | |
|---|---|---|---|---|---|
| At2g39800 | P5CS1 delta 1-pyrroline-5- | 2.6E−06 | 1.30 | 2.10 | 5.40 |
| | carboxylate synthetase A | | | | |
| At1g60590 | Polygalacturonase, putative | 5.0E−05 | 1.73 | 2.22 | 4.28 |
| At3g50400 | GDSL-motif lipase/hydrolase | 4.1E−07 | 1.94 | 2.95 | 4.23 |
| | family protein | | | | |
| At4g02280 | Sucrose synthase | 4.8E−07 | 1.70 | 2.00 | 3.40 |
| At3g57010 | Strictosidine synthase family | 1.8E−05 | 1.16 | 2.73 | 3.37 |
| | protein | | | | |
| At1g04220 | Beta-ketoacyl-CoA synthase, | 1.4E−05 | 1.86 | 1.96 | 3.08 |
| | putative | | | | |
| At3g25820 | ATTPS-CIN monoterpene | 2.3E−04 | 1.13 | 2.76 | 2.96 |
| | 1,8-cineole synthase | | | | |
| At1g74460 | GDSL-motif lipase/hydrolase | 1.7E−07 | 1.65 | 2.41 | 2.87 |
| | family protein | | | | |
| At5g20830 | SUS1 sucrose synthase | 1.4E−04 | −1.10 | 2.20 | 2.40 |
| At1g61810 | Glycosyl hydrolase family 1 | 1.1E−04 | 1.42 | 1.97 | 2.36 |
| | protein | | | | |

Transport

| | | | | | |
|---|---|---|---|---|---|
| At2g41190 | Amino acid transporter | 0.00922 | 1.51 | 2.00 | 4.46 |
| At5g13900 | Protease inhibitor/seed | 1.6E−04 | 1.86 | 2.09 | 3.95 |
| | storage/lipid transfer protein | | | | |
| At1g62510 | Protease inhibitor/seed | 6.0E−08 | 1.25 | 2.20 | 3.40 |
| | storage/lipid transfer protein | | | | |
| At2g18370 | Protease inhibitor/seed | 4.8E−05 | 1.44 | 2.12 | 2.61 |
| | storage/lipid transfer protein | | | | |

Peroxidases

| | | | | | |
|---|---|---|---|---|---|
| At1g68850 | Peroxidase | 0.00662 | 1.66 | 2.08 | 4.12 |

LEAs/Dehydrins

| | | | | | |
|---|---|---|---|---|---|
| At3g17520 | Late embryogenesis | 0.00250 | 1.91 | 5.97 | 10.12 |
| | abundant protein | | | | |
| At5g06760 | Late embryogenesis | 2.9E−07 | 1.35 | 2.82 | 4.53 |
| | abundant protein | | | | |
| At3g50980 | Dehydrin, putative | 0.00084 | 1.43 | 2.00 | 3.03 |

Transcription Factor

| | | | | | |
|---|---|---|---|---|---|
| At5g43840 | HSFA6A heat shock | 3.3E−07 | 1.73 | 1.96 | 9.38 |
| | transcription factor protein | | | | |
| At2g46680 | ATHB-7 homeobox-leucine | 1.9E−09 | 1.82 | 2.79 | 6.67 |
| | zipper protein 7 | | | | |
| At1g75030 | ATLP-3 pathogenesis- | 2.1E−05 | 1.81 | 2.28 | 5.09 |
| | related thaumatin family | | | | |
| | protein | | | | |

Other

| | | | | | |
|---|---|---|---|---|---|
| At5g13170 | Nodulin MtN3 family protein | 4.1E−07 | 1.79 | 2.85 | 5.19 |
| At2g47770 | Benzodiazepine receptor- | 3.8E−09 | 1.64 | 3.69 | 5.05 |
| | related | | | | |
| At1g64110 | AAA-type ATPase family | 3.4E−10 | 1.52 | 2.46 | 4.66 |
| | protein | | | | |
| At2g39510 | Nodulin MtN21 family protein | 5.7E−11 | 1.93 | 2.70 | 3.78 |
| At5g12420 | Expressed protein | 1.9E−05 | 1.85 | 2.67 | 3.50 |
| At5g41040 | Transferase family protein | 6.5E−05 | 1.70 | 1.97 | 3.03 |
| At1g56320 | NCED4 9-cis- | 2.0E−07 | 1.85 | 2.19 | 2.58 |
| | expoxycarotenoid | | | | |
| | dioxygenase, putative | | | | |
| At4g19170 | | 1.5E−05 | 1.39 | 2.22 | 2.51 |

Genes that do not respond to sorbitol stress in the athk1 null mutant were also identified (Table 3). As well, genes that show greater expression in the 35S ATHK1 mutant than wild-type in response to sorbitol stress were identified (Table 3). These genes are predicted to be immediate downstream targets of the ATHK1 signal transduction pathway. Notably, genes for proline and sucrose synthesis as well as other carbohydrate biosynthesis enzymes are overexpressed in the 35S ATHK1 mutant, suggesting, although by no means confirming, a possible mechanism involving increased production of osmolytes as a means of increased stress tolerance.

The above examples indicate that impairing the ATHK1 gene results in plant tissue that is defective in the ability to withstand osmotic stress, whereas increasing levels of the ATHK1 gene product results in plants with increased ability to withstand osmotic stress. RNA expression studies indicating that ATHK1 is most abundant in roots are consistent with the suggested role for ATHK1 in osmotic stress tolerance during germination and early plant development.

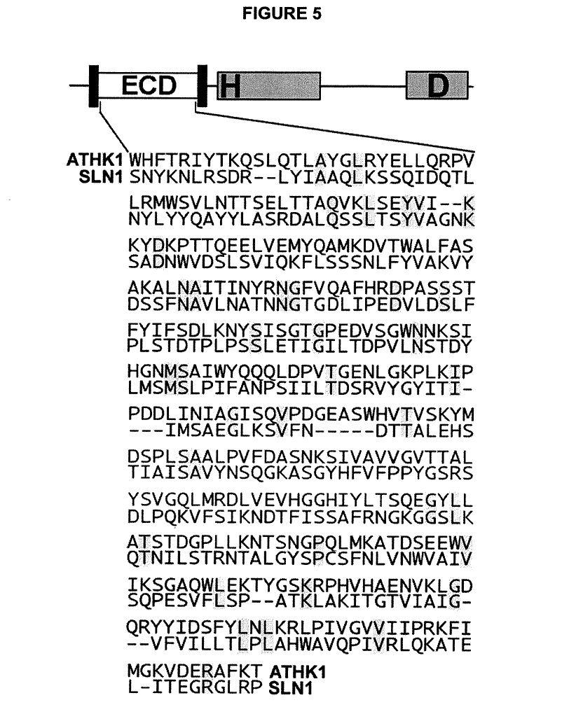

FIG. 5 illustrates how the ATHK1 extracellular domain (ECD) shows no significant homology to that of the yeast osmosensor histidine kinase SLN1. Identical residues in the two ECDs are shaded grey. The histidine residue (H) in the kinase domain and the aspartate residue (D) in the receiver domain are also indicated.

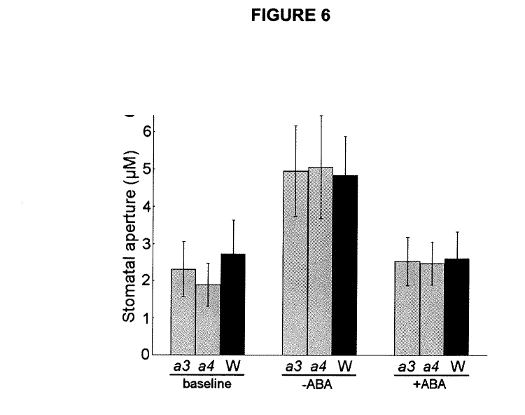

FIG. 6 is a graph showing stomatal response of ATHK1 alleles. Mature adult rosette leaves from wild-type (W; dark grey), athk1-3 (a3; light grey), and athk1-4 (a4; light grey) were initially held in the dark (baseline). Stomata were then induced to open with light in the presence or absence of ABA. Bars represent the mean of the average stomatal aperture for three experiments (2 leaves per experiment, with 20 stomatal aperture measurements per leaf). Error indicated is the standard deviation of the experimental means.

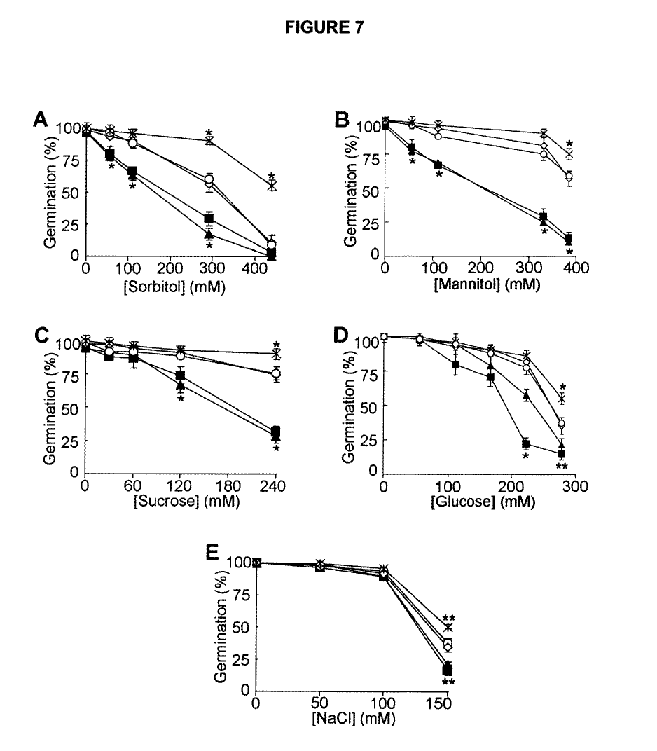

FIG. 7 is a graph showing altered osmotic and ionic sensitivity of ATHK1 alleles during germination. Wild-type (open diamonds), athk1-3 (closed squares), athk1-4 (closed triangles), athk1/ATHK1 rescued (open circles), and a 35S:ATHK1 overexpressor (asterisks) from matched seed lots were scored for germination on the indicated concentrations of sorbitol (A.), mannitol (B.), sucrose, (C.), glucose (D.), and NaCl (E.). The percent germination after five days of stress treatment is shown. Each value represents the mean percent germination for at least four replicates of at least 100 seeds. Error bars represent the SE. Asterisks represent significance based on a two-tailed t-test (*p<0.01, **p<0.05).

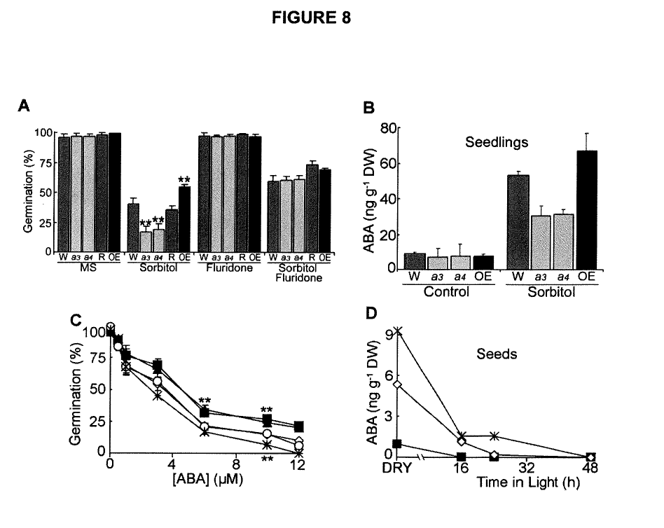

FIG. 8 illustrates how ATHK1 alleles display altered abscisic acid (ABA) phenotypes. Wild-type (W; dark grey bars and open diamonds), athk1-3 (a3; light grey bars and closed squares), athk1-4 (a4; light grey bars and closed triangles), athk1/ATHK1 rescued (R; dark grey bars and open circles), and a 35S:ATHK1 overexpressor (OE; black bars and stars) were used for these assays. Error bars represent the SE. Asterisks represent significance based on a two-tailed t-test (*p<0.01; **p<0.05). FIG. 8A: Effect of the ABA inhibitor fluridone on osmotic sensitivities of ATHK1 alleles. Seeds from matched lots were germinated on MS media ±300 mM sorbitol and ±100 μM fluridone. Each value represents the mean percent germination after five days of stress treatment for five replicates of at least 50 seeds. FIG. 8B: ABA levels in vegetative tissues of wild-type and ATHK1 mutants. Five-day old seedlings were exposed to water ±300 mM sorbitol for 16 h. Each value represents the mean ABA level of three independent biological replicates. FIG. 8C: Altered ABA sensitivities in germination of ATHK1 alleles. Seeds from matched lots were germinated on MS media ±ABA. Each value represents the mean percent germination after five days of ABA treatment for four replicates of 100 seeds. FIG. 8D: ABA levels in wild-type and ATHK1 mutant seeds. Samples were collected from dry seeds (0 hours) and seeds after 16, 24, and 48 hours of inhibition. Each value represents the mean ABA level of four independent biological replicates.

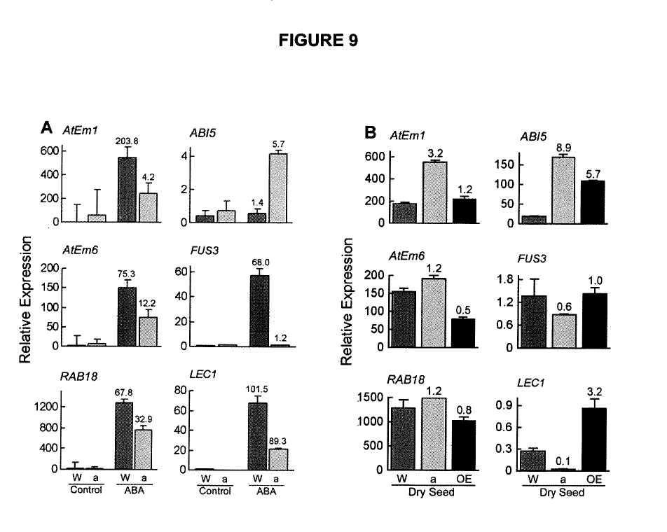

FIG. 9 shows graphs illustrating altered expression of ABA-regulated seed transcripts in ATHK1 mutants. Expression levels in wild-type (W; dark grey), athk1-3 (a; light grey), and a 35S:ATHK1 overexpressor (OE; black) were assayed by quantitative RT-PCR. All values were normalized to the actin control ACT2 gene. Error bars represent the SE. FIG. 9A is expression of the ABA-upregulated genes AtEm1, AtEm6, RAB18, ABI5, FUS3, and LEC1. Five-day old seedlings were exposed to water ±1 μM ABA for 16 hours. Bars represent the relative mean expression level from five PCR reactions. Printed numbers represent the fold change over the same genotype non-stressed control sample. FIG. 9B is expression of the seed transcripts AtEm1, AtEm6, RAB18, ABI5, FUS3, and LEC1 in dry seeds. Bars represent the relative mean expression level from three PCR reactions. Printed numbers represent the fold change over the wild-type.

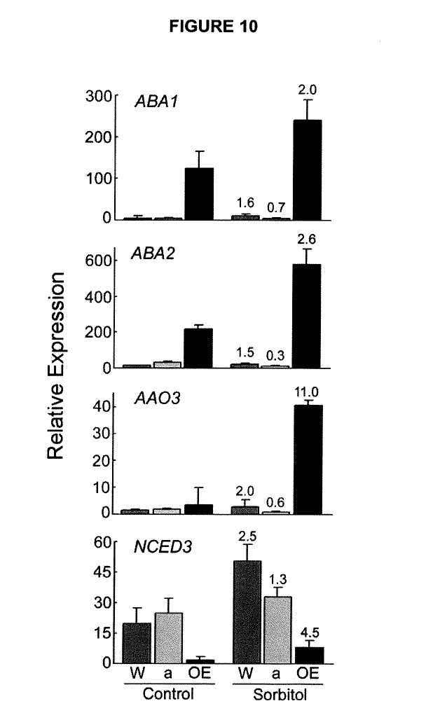

FIG. 10 shows graphs illustrating altered expression of ABA biosynthetic genes in ATHK1 mutants. Expression levels of the ABA biosynthetic genes ABA1, ABA2, AAO3, and NCED3 in wild-type (W; dark grey), athk1-3 (a; light grey), and a 35S:ATHK1 overexpressor (OE; black) were assayed by quantitative RT-PCR. Five-day old seedlings were exposed to water ±300 mM sorbitol for 16 hours. All values were normalized to the actin control ACT2 gene. Bars represent the relative mean expression level from five PCR reactions. Printed numbers represent the fold change over the same genotype non-stressed control sample. Error bars represent the SE.

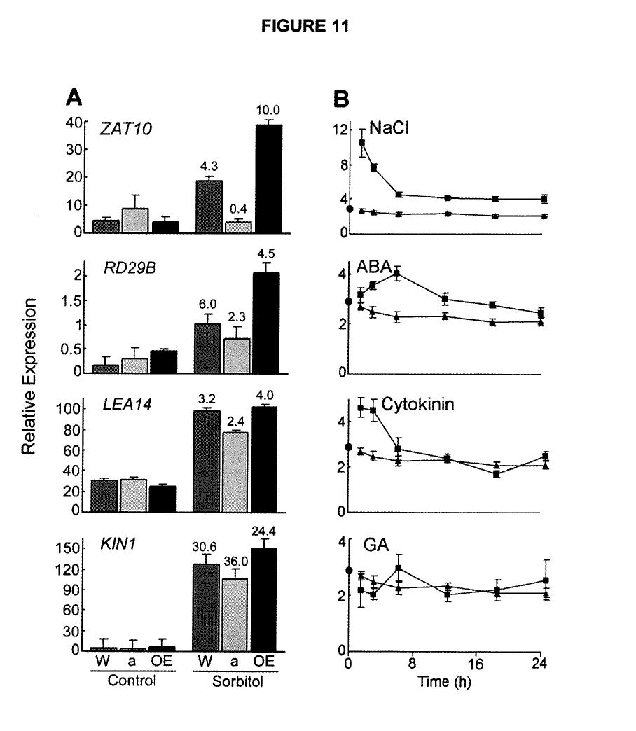

FIG. 11 shows graphs illustrating altered expression of osmotic stress-responsive genes in ATHK1 mutants and expression of ATHK1 in response to stress. Expression levels were assayed by quantitative RT-PCR. All values were normalized to the actin control ACT2 gene. Error bars represent the SE. FIG. 11A is expression of the osmotic stress-responsive genes ZAT10, RD29B, KIN1, and LEA14. Wild-type (W; dark grey), athk1-3 (a; light grey), and 35S:ATHK1 overexpressor (OE; black) five-day old seedlings were exposed to water ±300 mM sorbitol for 16 hours. Bars represent the relative mean expression level from five PCR reactions. Printed numbers represent the fold change over the same genotype, non-stressed control sample. FIG. 11B is expression of the ATHK1 transcript after stress. Ten-day old wild-type seedlings were exposed to water ±100 mM NaCl, 1 μM abscisic acid (ABA), 1 μM cytokinin (6-BAP), or 1 μM gibberellin (GA) (closed squares) for 24 hours. Samples of whole-seedling tissue were collected at 0 h, 1.5 h, 3 h, 6 h, 12 h, 18 h, and 24 h. The control (0 h) levels of ATHK1 are shown with a closed circle. Values represent the relative mean expression level from four PCR reactions.

It is to be understood that this invention is not limited to the particular compounds, devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters normally encountered in molecular biology and plant physiology, obvious to those skilled in the art, are within the scope of this invention. All publications, patents, and patent applications cited herein are incorporated by reference in their entirety for all purposes to the same extent as if each individual publication, patent, or patent application were specifically and individually indicated to be so incorporated by reference.

What is claimed is:

1. An expression cassette for plants comprising a promoter operably linked to a heterologous ATHK1 polynucleotide, or a complement thereof, encoding a polypeptide at least 95% identical to SEQ ID NO:26, wherein the polynucleotide encodes a polypeptide that confers drought resistance in a plant.

2. The expression cassette of claim 1, wherein the ATHK1 polynucleotide, or the complement thereof, is at least 95% identical to SEQ ID NO:25.

3. The expression cassette of claim 1, wherein the ATHK1 polynucleotide, or the complement thereof, comprises positions from about 208 to about 3831 of SEQ ID NO:25.

4. The expression cassette of claim 1, wherein the promoter is constitutive.

5. The expression cassette of claim 1, wherein the promoter is tissue-specific.

6. The expression cassette of claim 1, wherein the promoter is inducible.

7. A plant expression vector comprising the expression cassette of claim 1.

8. A host cell comprising the expression cassette of claim 1.

9. The host cell of claim 8, which is a plant cell.

10. A transgenic plant comprising an isolated polynucleotide encoding:
a) a polypeptide comprising an amino acid sequence that is at least 95% identical to SEQ ID NO:26, or
b) a polypeptide comprising at least 1147 amino acids of SEQ ID NO:26,
wherein the polypeptide confers drought resistance in the transgenic plant.

11. The transgenic plant of claim 10, which is a dicotyledon.

12. The transgenic plant of claim 10, which is a brassicaceous plant.

13. A method of producing a transgenic plant with enhanced drought resistance, the method comprising introducing into the plant an isolated polynucleotide encoding:
a) a polypeptide comprising an amino acid sequence that is at least 95% identical to SEQ ID NO:26, or
b) a polypeptide comprising at least 1147 amino acids of SEQ ID NO:26,
wherein the polypeptide confers drought resistance in the transgenic plant.

14. A plant with enhanced drought resistance, the plant comprising a recombinant expression cassette for plants comprising a promoter operably linked to a polynucleotide encoding a polypeptide at least 95% identical to SEQ ID NO:26.

15. The plant of claim 14, wherein the polynucleotide encoding the polypeptide is at least 95% identical to SEQ ID NO:25.

16. The plant of claim 14, wherein the promoter is constitutive.

17. The plant of claim 14, wherein the promoter is tissue-specific.

18. The plant of claim 14, wherein the promoter is inducible.

19. A method of producing a transgenic plant with enhanced drought resistance, the method comprising introducing into the plant a recombinant expression cassette for plants comprising a polynucleotide encoding a polypeptide at least 95% identical to SEQ ID NO:26.

20. A method of growing a crop with enhanced drought resistance comprising introducing into the crop an expression cassette for plants comprising a promoter sequence operably linked to a heterologous ATHK1 polynucleotide, or a complement thereof, encoding a polypeptide at least 95% identical to SEQ ID NO:26, wherein the polynucleotide encodes a polypeptide that confers drought resistance in the crop.

21. The method of claim 20, wherein the crop is a dicotyledon.

22. The method of claim 20, wherein the crop is a brassicaceous plant.

23. A transgenic plant which overexpresses a polynucleotide encoding a polypeptide at least 95% identical to SEQ ID NO:26 in said plant as compared to a non-transformed plant, wherein said overexpression causes increased drought resistance in the plant.

24. The transgenic plant of claim 23, which is a dicotyledonous plant.

25. Seed obtained by growing the transgenic plant of claim 23.

26. A transgenic plant which underexpresses a polynucleotide encoding a polypeptide at least 95% identical to SEQ ID NO:26 in said plant as compared to a non-transformed plant, wherein said underexpression causes decreased drought resistance in the plant.

27. The transgenic plant of claim 27, which is a dicotyledonous plant.

28. Seed obtained by growing the transgenic plant of claim 27.
